(19) United States
(12) Patent Application Publication
(10) Pub. No.: US 2012/0065084 A1
(43) Pub. Date: Mar. 15, 2012

(54) METHODS AND TOOLS FOR PREDICTING THE EFFICIENCY OF ANTHRACYCLINES IN CANCER

(75) Inventors: Christos Sotiriou, Bruxelles (BE); Christine Desmedt, Meise (BE)

(73) Assignee: UNIVERSITE LIBRE DE BRUXELLES, Bruxelles (BE)

(21) Appl. No.: 13/264,726

(22) PCT Filed: Apr. 16, 2010
(86) PCT No.: PCT/EP2010/055067

§ 371 Date: Oct. 14, 2011

(51) Int. Cl. C40B 30/00; C40B 40/06; C07H 15/252; C40B 40/08

(52) U.S. Cl. 506/7; 506/17; 506/16; 536/6.4

(57) ABSTRACT

A gene set, kit and method predict the efficiency of anthracyclines-based treatment of breast cancer.

### FIELD OF THE INVENTION

The present invention is related to methods and tools (gene set and kit or device) to predict the efficiency of an anthracycline(s)-based regimen in mammal subjects affected by an hyperproliferative disorder (cancer), especially human patients affected by this disorder, especially human patients affected by Breast cancer (BC).

### BACKGROUND OF THE INVENTION

Breast cancer (BC) is the most common cancer in women in Western countries.

Breast cancer is an heterogeneous disease that can be subdivided into subgroups depending on markers.

For instance, oestrogen receptor (ER) positive status is associated with a better outcome and may predict for a response to hormone treatments.

Her2 (ERBB2, neu) overexpression is associated to a worse outcome, but Her2-specific treatments, such as administration of monoclonal antibodies (trastuzumab or herceptin), may be beneficial for those patients.

Anthracyclines-based regimens are among the most active chemotherapies in Breast Cancer. However, their clinical use is associated with rare but severe toxicities, such as long-term hematological disorders (myelodisplastic syndrome and leukemia) and cardiac heart failure. Also the efficacy of anthracyclines appears to be restricted to a subset of the Breast Cancer patient population. Therefore, the identification of molecular markers that could predict a response of breast tumors to anthracyclines-based chemotherapy remains a priority.

Cell lines studies have suggested that cells with high amounts of Topoisomerase II alpha (TOP2A) might be more sensitive to anthracyclines. Several groups have investigated this hypothesis during the last decade and controversial results regarding TOP2A amplification/expression and response to anthracyclines in Breast Cancer patients have been reported.

Several studies, mostly retrospective, hypothesized that the amplification/overexpression of TOP2A might influence a response to an anthracyclines therapy, often with contradictory results.

It is known that a TOP2A overexpression or amplification were not predictive of response to neo-adjuvant anthracyclines-based chemotherapy (Petit et al., 2004, Eur J Cancer; 40:205-11).

TOP2A quantification at the gene level and at the protein level are not correlated (Di Leo et al., 2008, Eur. J. of Cancer, 44, 2791-2798).

Some studies conclude that the prediction is restricted to the measure of the protein expression of TOP2A, while for other the best results are achieved by the monitoring of DNA amplification. For instance, Bartlett et al (J. Clin. Oncol, 2008, 31, 5027-5035) concludes that TOP2A gene amplification measures by FISH is not predictive of response to anthracyclin treatments. Knoop et al. (2005), J. Clin Oncol, 23, 7483-7490 conclude that both amplification and deletion of TOP2A is predictive for a response to anthracyclines.

These contrasting results reported in the literature regarding the predictive value of TOP2A may be explained by different reasons: the chemotherapy regimen used (monotherapy versus polychemotherapy, anthracyclines-based or including other drugs, such as taxanes) given in the neo-adjuvant or adjuvant setting, the diversity of treated patients included in these different studies, the heterogeneity in the assessment of a patient response (clinical, radiological and/or pathological response) and the different methods and levels of TOP2A evaluation.

For instance, FISH results appear to be linked to a high variability between different test centres and up to 31% of discordance between local and central labs has been reported for FISH measurement of Her2 and/or TOP2A (Di Leo A et al, Cancer Res., 69 suppl (abstr 705) 2009).

### AIMS OF THE INVENTION

The present invention aims to provide new detection methods and tools that do not present the drawbacks of the state of the art.

The present invention aims to provide such methods and tools that improve the prediction of a response to antracyclines (-based) regimens in breast cancer patients.

### SUMMARY OF THE INVENTION

The present invention relates to a gene set representing TOP2A index.

Advantageously, the TOP2A index is measured by a mRNA quantification of these selected genes.

Possibly, the genes corresponding to the gene set representing the Top2A index are isolated.

Advantageously, the gene set of the invention (representing TOP2A index) comprises (or consist of) 1, 2, 3, 4, 5, 6, 7, 8, 9, 10, 11, 12, 13, 14, 15, 16, 17, 18, 19, 20, 21, 22 or all the genes mentioned in Table 1.

The present invention relates also to a gene set (representing TOP2A index) consisting of TOP2A (Gene ID: 7153) and of 1, 2, 3, 4, 5, 6, 7, 8, 9, 10, 11, 12, 13, 14, 15, 16, 17, 18, 19, 20, 21, 22 or all the genes mentioned in Table 1.

The present invention further relates to a gene set (representing TOP2A index) consisting of RARA (Gene ID: 5914) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of THRA (Gene ID: 7067) and further comprising 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of CDC6 (Gene ID: 990) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of GSDM1 (Gene ID: 284110) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of PSMD3 (Gene ID: 5709) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of CSF3 (Gene ID: 1440) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of MED24 (Gene ID: 9862) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of SNORD124 (Gene ID: 100113390) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of NR1D1 (Gene ID: 9572) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of TRNASTOP-UCA (Gene ID: 100126534) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of MSL-1 (Gene ID: 339287) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of CASC3 (Gene ID: 22794) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of RAPGEFL1 (Gene ID: 51195) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of WIPF2 (Gene ID: 147179) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of LOC100131821 (Gene ID: 100131821) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of GJD3 (Gene ID: 125111) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of LOC390791 (Gene ID: 390791) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of LOC728207 (Gene ID: 728207) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of IGFBP4 (Gene ID: 3487) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of TNS4 (Gene ID: 84951) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of CCR7 (Gene ID: 1236) and of 1, 2, 3, 4, 5, 6, 7, 8, 9, 10, 11, 12, 13, 14, 15, 16, 17, 18, 19, 20, 21 or all the genes of Table 1.

The present invention also relates to a gene set (representing TOP2A index) consisting of SMARCE1 (Gene ID: 6605) and of 1, 2, 3, 4, 5, 6, 7, 8, 9, 10, 11, 12, 13, 14, 15, 16, 17, 18, 19, 20, 21 or all the genes of Table 1.

Preferably, the present invention relates to a gene set (representing TOP2A index) comprising (or consisting of) two or three genes selected from the group consisting of CDC6 (Gene ID: 990), THRA (Gene ID: 9572), RARA (Gene ID: 5914) and TOP2A (Gene ID: 7153).

Preferably, the present invention relates to a gene set (representing TOP2A index) comprising (or consisting of) two or three genes selected from the group consisting of CDC6 (Gene ID: 990), THRA (Gene ID: 9572), RARA (Gene ID: 5914) and TOP2A (Gene ID: 7153) and 1, 2, 3, 4, 5, 6, 7, 8, 9, 10, 11, 12, 13, 14, 15, or all the genes of Table 1.

Advantageously, the present invention relates to a gene set (representing TOP2A index) comprising (or consisting of) CDC6 (Gene ID: 990), RARA (Gene ID: 5914) and WIPF2 (Gene ID: 147179).

Preferably, the present invention relates to a gene set (representing TOP2A index) comprising (or consisting of) THRA (Gene ID: 9572), CDC6 (Gene ID: 990), RARA (Gene ID: 5914) and TOP2A (Gene ID: 7153).

Preferably the present invention relates to a gene set (representing TOP2A index) comprising (or consisting of) THRA (Gene ID: 9572), CDC6 (Gene ID: 990), RARA (Gene ID: 5914) and TOP2A (Gene ID: 7153) and further comprising (consisting of) 1, 2, 3, 4, 5, 6, 7, 8, 9, 10, 11, 12, 13, 14, 15, 16, 17, 18, or all the genes of Table 1.

The present invention relates to a gene set (representing TOP2A index) that may further comprise 1, 2, 3, 4, 5, 6, 7, 8, 9, 10, 15, 20, 25, 30, 35, 40, 45, 50, 55, 60, 65, 70, 75 or all the genes of Table 5 and/or Table 6.

Alternatively, the present invention relates to a gene set comprising (or consisting of) 1, 2, 3, 4, 5, 6, 7, 8, 9, 10, 15, 20, 25, 30, 35, 40, 45, 50, 55, 60, 65, 70, 75 or all the genes of Table 5.

Alternatively, the present invention relates to a gene set comprising (or consisting of) 1, 2, 3, 4, 5, 6, 7, 8, 9, 10, 15, 20, 25, 30, 35, 40, 45, 50, 55, 60, 65, 70, 75 or all the genes of Table 6.

The present invention relates to a gene set (representing TOP2A index) that further comprises 1, 2, 3, 4, 5, 6, 7, 8, 9, 10 or all the genes of Table 10.

The present invention relates to a gene set (representing TOP2A index) that further comprises 1, 2, 3, 4, 5, 6, 7, 8, 9, 10 or all the genes of Table 11.

The present invention relates to a gene set (representing TOP2A index) that further comprises 1, 2, 3, 4, 5, 6, 7, 8, 9, 10 or all the genes of Table 12.

The present invention relates to a gene set (representing TOP2A index) that further comprises 1, 2, 3, 4, 5, 6, 7, 8, 9, 10 or all the genes of Table 13.

The present invention relates to a gene set (representing TOP2A index) that further comprises 1, 2, 3, 4, 5, 6, 7, 8, 9, 10 or all the genes of Table 14.

The present invention relates to a gene set (representing TOP2A index) that further comprises 1, 2, 3, 4, 5, 6, 7, 8, 9, 10 or all the genes of Table 15.

The present invention relates to a gene set (representing TOP2A index) that further comprises 1, 2, 3, 4, 5, 6, 7, 8, 9, 10 or all the genes of Table 16.

Most preferably, the present invention relates to a gene set (representing TOP2A index, the said genes representing Top2A index preferably (consisting of or) comprising CDC6, RARA and WIPF2) that further comprises FAM64A, KIF4A, NCAPH and STIL.

Advantageously, the gene set (representing TOP2A index) comprises (or consists of) CDC6, RARA, WIPF2, FAM64A, KIF4A, NCAPH and STIL and further comprises 1, 2, 3, 4, 5, 6, 7, 8, 9 or 10 genes of Table 10 and/or 11.

The present invention relates to a gene set (representing TOP2A index) that further comprises 1, 2, 3, 4, 5, 6, 7, 8, 9, 10 or all the genes of Table 17.

The present invention relates to a gene set (representing TOP2A index) that further comprises 1, 2, 3, 4, 5, 6, 7, 8, 9, 10 or all the genes of Table 18.

The present invention further relates to a diagnostic kit or device comprising capture probes (nucleotides sequences or proteinic probes such as antibodies, nanobodies or hypervariable portions thereof) possibly fixed upon a solid support, (such as a multiwell plate or glass side) able to detect specifically, the expression of (complementary) target genes (being complementary by hybridization to the capture probes) (or their corresponding proteins encoded by these genes) of the set of the invention (representing TOP2A index), and/or the gene set of Tables 5 and/or 6 and possibly other means used for real time PCR analysis.

Preferably, the kit or device according to the invention comprises means for real time PCR, preferably means for qRT-PCR.

Advantageously, the kit or device of the invention is a computerized system comprising

a bio-assay module configured for detecting a gene expression from a tumor sample (preferably a breast tumor sample) based upon the gene set or kit according of the invention and

a processor module configured to calculate expression of these genes or protein synthesis of these genes and to generate a risk assessment for the tumor (preferably a breast tumor) sample.

Another aspect of the invention concerns a method for a prediction (prognosis or prognostic) of cancer in mammal subject, which comprises the step of measuring gene expression in a (breast) tumor sample obtained from the said mammal subject, by putting into contact nucleotide sequences (or proteins) obtained from this tumor sample with the gene set, the kit or the device according to the invention and possibly generating a risk assessment for the said tumor sample by designating the outcome of a regimen comprising administration to the said mammal subject, of a sufficient amount of one or more Anthracycline compound(s) and by selecting the adequate compound to be administrated to this patient, preferably the sufficient amount of one or more anthracycline compound(s).

Preferably in this method, the patient is a human patient, suffering from a cancer selected from the group consisting of ovary cancer, breast cancer, lung cancer, cancer of the womb, cancer of the bladder, cancer of the stomach, sarcomas, thyroid cancer, leukaemia, Hodgkin's lymphoma and multiple myeloma (preferably a breast cancer, more preferably a ER− breast cancer still more preferably a ER− Her2+ breast cancer).

Preferably, the gene set, kit and device of the invention predicts the response of regimen consisting essentially into the administration to the tested patient of one or more Anthracycline compound(s), possibly combined with a simultaneous or a separated administration of another anti-tumoral therapeutic treatment or compound.

Another aspect of the invention is related to an Anthracycline compound or a mixture of Anthracycline compounds (regimen) for use in the treatment (and/or the prevention) of cancer patients determined (as measured) by the gene set, kit or method of the invention.

Advantageously, Anthracycline compound or a mixture of Anthracycline compounds (regimen) is for use in the treatment (and/or prevention) of a cancer selected from the group consisting of ovary cancer, breast cancer, lung cancer, cancer of the womb, cancer of the bladder, cancer of the stomach, sarcomas, thyroid cancer, leukemias, Hodgkin's lymphoma and multiple myeloma, having (increased) index (as measured) according to Table 5 and/or a worse prediction (prognosis) assessed by the (prediction or prognostic) method of the invention.

Alternatively, Anthracycline compound or a mixture of Anthracycline compounds (regimen) is for use in the treatment (and/or prevention) of a cancer selected from the group consisting of ovary cancer, breast cancer, lung cancer, cancer of the womb, cancer of the bladder, cancer of the stomach, sarcomas, thyroid cancer, leukemias, Hodgkin's lymphoma and multiple myeloma, having (increased) index (as measured) according to Table 6 and/or a worse prediction (prognosis) assessed by the prediction (prognostic) method of the invention.

Preferably, Anthracycline compound or a mixture of Anthracycline compounds (regimen) is for use in the treatment (and/or prevention) of (breast) cancer, preferably ER− breast cancer, more preferably a ER− Her2− breast cancer having (increased) index (as measured) according to the prediction (prognostic) method of the invention.

Alternatively, Anthracycline compound or a mixture of Anthracycline compounds (regimen) is for use in the treatment (and/or prevention) of (an ER− Her2+ breast) cancer having (increased) index (as measured) according to the prediction (prognostic) method of the invention.

Another aspect of the invention is related to a chemotherapeutic (anthracycline) compound (or regimen) for use in the treatment (and/or the prevention) of breast cancer having a specific (increased) index (as measured) according to Table 10 and/or Table 11, possibly combined with a specific (increased) TOP2A index (as measured according to Table 1), being preferably THRA (Gene ID: 9572), CDC6 (Gene ID: 990), RARA (Gene ID: 5914) and TOP2A (Gene ID: 7153).

Another aspect of the invention is related to a chemotherapeutic (anthracycline) compound (or regimen) for use in the treatment (and/or the prevention) of breast cancer having a specific index (as measured) according to Table 12 and/or Table 13 and/or Table 16, possibly combined with a specific (increased) TOP2A index (as measured according to Table 1), being preferably THRA (Gene ID: 9572), CDC6 (Gene ID: 990), RARA (Gene ID: 5914) and TOP2A (Gene ID: 7153).

A related aspect of the invention is a chemotherapeutic (anthracycline) compound (or regimen) for use in the treatment (and/or the prevention) of breast cancer having a specific (increased) index (as measured) according to Table 10 and/or Table 11, possibly combined with a specific (increased) index (as measured) according to Table 16, possibly combined with a specific (reduced) index (as measured) according to Table 12 and/or Table 13 possibly combined with a specific (increased) TOP2A index (as measured according to Table 1), being preferably THRA (Gene ID: 9572), CDC6 (Gene ID: 990), RARA (Gene ID: 5914) and TOP2A (Gene ID: 7153) or alternatively being CDC6, RARA and WIPF2.

A last aspect of the invention is a method of treatment of patients comprising the steps of
measuring gene expression (using one or several the gene sets of the Tables 1, 5-18) in a (breast) tumor sample obtained from the said mammal subject, by putting into contact nucleotide sequences with the gene set, the kit or the device according to the invention;
possibly generating a risk assessment for the said tumor sample by designating the outcome of a regimen comprising administration to the said mammal subject, of a sufficient amount of one or more Anthracycline compound(s) and
selecting the adequate compound to be administrated to this patient, preferably the sufficient amount of one or more anthracycline compound(s).

TABLE 1
genes composing TOP2A index

| Symbol | GeneID | Cyto |
|---|---|---|
| RARA | 5914 | 17q21 |
| CDC6 | 990 | 17q21.3 |
| THRA | 7067 | 17q11.2 |
| GSDM1 | 284110 | 17q12 |
| PSMD3 | 5709 | 17q12-q21.1 |
| CSF3 | 1440 | 17q11.2-q12 |
| MED24 | 9862 | 17q21.1 |
| SNORD124 | 100113390 | 17q21.1 |
| NR1D1 | 9572 | 17q11.2 |
| TRNASTOP-UCA | 100126534 | 17q21.1 |
| MSL-1 | 339287 | 17q21.1 |
| CASC3 | 22794 | 17q11-q21.3 |
| RAPGEFL1 | 51195 | 17q21.1-q21.2 |
| WIPF2 | 147179 | 17q21.2 |
| LOC100131821 | 100131821 | 17q21.2 |
| GJD3 | 125111 | 17q21.2 |
| LOC390791 | 390791 | 17q21.2 |
| LOC728207 | 728207 | 17q21.2 |
| IGFBP4 | 3487 | 17q12-q21.1 |
| TNS4 | 84951 | 17q21.2 |
| CCR7 | 1236 | 17q12-q21.2 |
| TOP2A | 7153 | 17q21-17q22 |
| SMARCE1 | 6605 | 17q21.2 |

Note: RARA stands for retinoic acid receptor, alpha; CDC6 for cell division cycle 6 homolog (S. cerevisiae); THRA for thyroid hormone receptor, alpha (erythroblastic leukemia viral (v-erb-a) oncogene homolog, avian); GSDM1 for gasdermin 1; PSMD3 for proteasome (prosome, macropain) 26S subunit, non-ATPase, 3; CSF3 for colony stimulating factor 3 (granulocyte); MED24 for mediator complex subunit 24; SNORD124 for small nucleolar RNA, C/D box 124; NR1D1 for nuclear receptor subfamily 1, group D, member 1; TRNASTOP-UCA for transfer RNA opal suppressor (anticodon UCA); MSL-1 for male-specific lethal-1 homolog; CASC3 for cancer susceptibility candidate 3; RAPGEFL1 for Rap guanine nucleotide exchange factor (GEF)-like 1; WIPF2 for WAS/WASL interacting protein family, member 2; LOC100131821 for hypothetical protein LOC100131821; GJD3 for gap junction protein, delta 3, 31.9 kDa; LOC390791 for similar to peptidylprolyl isomerase A isoform 1; LOC728207 for similar to 60S ribosomal protein L23a; IGFBP4 for insulin-like growth factor binding protein 4; TNS4 for tensin 4; CCR7 for chemokine (C-C motif) receptor 7; TOP2A for DNA topoisomerase II and SMARCE1 for SWI/SNF related, matrix associated, actin dependent regulator of chromatin, subfamily e, member 1.

### DETAILED DESCRIPTION OF THE INVENTION

### Patient's Description

One hundred and forty-nine patients were included in the prospective TOP trial. Out of these patients, 89 received 4 cycles of anthracyclines every 3 weeks, 59 patients received 6 cycles of anthracyclines administered every 2 weeks (dose-dense scheme) and one was not treated according to the protocol due to ineligibility.

One hundred and thirty-two patients completed treatment as per protocol, 16 discontinued (2 due to disease progression, 2 due to consent withdraw, 3 due to adverse experience and 9 for another reason).

A pathological complete response (pCR) rate of 15% was obtained, 14% and 16% for patients treated with the 3-weekly and dose-dense scheme respectively. The patient and tumour baseline characteristics are illustrated in Table 2.

TABLE 2
Patient and tumor baseline characteristics

| | Patients (n = 149) |
|---|---|
| Patients registered | 149 |
| Case Report Forms | 149 |
| collected | |
| Median age (range) | 47 (27-68) |
| Age ≦50 | 91 |
| Age >50 | 58 |
| Missing | 0 |
| T size (at baseline) | |
| ≦2 cm | 21 |
| >2 and ≦5 cm | 105 |
| >5 cm | 5 |
| T4 | 17 |
| Missing | 1* |
| N status (at baseline) | |
| N0 | 74 |
| N1 | 68 |
| N2 | 3 |
| N3 | 3 |
| Missing | 1* |
| Histological type | |
| ductal | 139** |
| lobular | 2 |
| other | 8 |
| Missing | 1* |
| Histological grade*** | |
| G1 | 2 |
| G2 | 28 |
| G3 | 110 |
| Gx (unknown) | 9 |
| Missing | 1* |
| Type of surgery | |
| Mastectomy | 49 |
| Conservative | 88 |
| Other | 1 |

*1 patient was ineligible
**1 patient had multifocal tumour and both tumors were ductal
***1 patient had multifocal tumour with different grade

The age of the patient, the size and grade of the tumor and the nodal status were not associated with pCR. Ki67 protein expression (a proliferation marker) was not significantly associated with pCR, both when considering it as a continuous and binary variable (Table 3).

TABLE 3
Association between the clinical parameters, FISH
results and response to treatment (pCR = pathological
complete response)

| | | Nr of | | |
|---|---|---|---|---|
| | | patients | Nr of | |
| | | with no | patients | Results of |
| | | pCR | with pCR | test |
| Age | ≦50 years | 78 | 9 | κ = 0.09 |
| | >50 years | 45 | 10 | (p = 0.18) |
| Size | T1 | 20 | 1 | κ = 0.03 |
| | T2-T4 | 103 | 18 | (p = 0.21) |
| Nodes | N0 | 63 | 8 | κ = 0.04 |
| | N1-3 | 60 | 11 | (p = 0.46) |
| Grade | G1-G2 | 25 | 4 | κ = 0.00 |
| | G3 | 92 | 14 | (p = 0.93) |
| Ki67 | ≦25% | 22 | 2 | κ = 0.03 |
| | >25% | 80 | 14 | (p = 0.40) |
| HER2_FISH | Not | 67 | 7 | κ = 0.16 |
| | amplified | | | (p = 0.06) |
| | Amplified | 27 | 8 | |
| TOP2A_FISH | Not | 89 | 9 | κ = 0.39 |
| | amplified | | | (p = 3.10⁻⁵) |
| | Amplified | 5 | 6 | |
| TOP2A_FISH | Deleted | 13 | 3 | p = 0.001 |
| | Normal | 76 | 6 | |
| | Amplified | 5 | 6 | |

### Topoisomerase II α (TOP2A)

The prospective investigation of the predictive value of TOP2A for response to anthracyclines was the primary aim of this trial. Advantageously, all TOP2A evaluations were carried out in a blinded fashion: TOP2A gene, mRNA and protein evaluations were done independently.

Gene expression profiles could be obtained for 120 patients out of the 149 (81%). The ER mRNA levels were used to double-check the ER-negativity reported by IHC.

TOP2A was represented by 3 different Affymetrix probe-sets: 201 291_s_at, 201 292_at and 237 469_at. Since the probe-set 201 291_s_at showed the greatest variance, the inventors used that probe-set for further analyses.

Ninety-one samples had results both at DNA and mRNA level and a statistically significant correlation was observed between these results, both when considering FISH results as a continuous (Spearman rho=0.35, p=0.001) or as a discrete variable (p=4 10⁻⁴ when considered as amplified/non-amplified, and p=0.001 when considered as deleted/normal/amplified).

FISH results were available for 113 out of the 149 patients. Out of these samples, 36 were HER2 amplified (32%); TOP2A was amplified in 12 patients (11% of the global population) and deleted in 16 patients (14% of the population). Noteworthy, all TOP2A amplified and 85% of the TOP2A deleted samples were also HER2 amplified.

TOP2A measures by IHC were available for 120 patients. The median percentage of positively stained cells was 15% (range 0-90%). When considering TOP2A expression as a continuous variable, the inventors did not observe any correlation with FISH results (based on the 99 ER-negative samples available for this comparison).

The inventors were able to observe a small but statistically significant correlation between protein and mRNA levels (Spearman rho=0.23, p=0.02, based on 96 samples). However, when the inventors used the median as a cut point to define overexpression, they did not find any correlation between the protein and other measurements of TOP2A.

The inventors then assessed whether the different measurements of TOP2A were associated with common clinico-pathological parameters such as age at diagnosis, tumor size, nodal status and histological grade. Except a significant association between TOP2A mRNA levels and histological grade, high grade tumors presenting higher levels of TOP2A mRNA, no other associations were observed (Table 4).

TABLE 4
TOP2A gene, mRNA and protein levels with regard to the clinic-pathologic parameters

| | | | Size of tumor at | | |
|---|---|---|---|---|---|
| | | Age at diagnosis | diagnosis | Nodal status | Histological Grade |
| | | (≦50 yrs, >50 yrs) | (T1 vs T2, T3, T4) | (N0 vs others) | (G1, 2 vs G3) |
| Gene level | continuous | p = 0.40 | p = 0.29 | p = 0.17 | p = 0.90 |
| (FISH) | amplified/non-amplified | κ = 0.05, p = 0.45 | κ = 0.01, p = 0.71 | κ = 0.00, p = 0.95 | κ = 0.01, p = 0.70 |
| | deleted/normal/amplified | p = 0.74 | p = 0.70 | p = 0.95 | p = 0.92 |
| mRNA level | Continuous | P = 0.43 | p = 0.56 | p = 0.70 | p = 0.03 |
| (Affymetrix) | Binary* | κ = −0.14, p = 0.13 | κ = 0.02, p = 0.79 | κ = −0.03, p = 0.71 | κ = 0.13, p = 0.08 |
| Protein level | Continuous | p = 0.51 | p = 0.54 | p = 0.75 | p = 0.43 |
| (IHC) | Binary* | κ = 0.01, p = 0.87 | κ = 0.00, p = 0.97 | κ = 0.00, p = 0.98 | κ = 0.07, p = 0.44 |

TOP2A amplification was found to be predictive of response (kappa=0.39, p=3 10⁻⁵) to anthracyclines. Indeed, as shown in Table 3, 55% of the patients with TOP2A amplified tumors responded to the anthracyclines therapy as opposed to 9% of the patients whose tumors had no TOP2A amplification. Also, a higher proportion of HER2-positive patients presented a pCR (23%) compared to HER2-negative patients (10%), however this difference did not reach statistical significance (kappa=0.16, p=0.06).

In this study, TOP2 mRNA levels (considered as a continuous variable or as a binary variable with the median as cut point) and TOP2A protein levels were not associated with response to therapy.

However, when carrying subgroup analyses according to the HER2 mRNA status (cfr methods), the inventors observed a C-index of 0.84 [95% confidence interval (CI): 0.70-0.97), p=2.10⁻⁷] for TOP2A mRNA, which means that a C-index of 0.84 can be interpreted that the probability that a patient who has a pathological complete response, has a higher TOP2A mRNA value than a patient who has no pathological complete response is of at least 84%.

### The TOP2A Amplicon

In order to better represent the TOP2A amplicon and to investigate whether other genes located closely to TOP2A might also be important in defining response to anthracyclines, the inventors developed different indices based on the averaged sum of the expression values of at least 2 genes located in the TOP2A amplicon.

By using a TOP2A index of 4 mRNA (THRA (Gene ID: 9572), CDC6 (Gene ID: 990), RARA (Gene ID: 5914) and TOP2A (Gene ID: 7153)), the inventors observed a C-index of 0.62 [95% CI: 0.46-0.78, p=0.06].

The inventors observed that the expression profile of the genes composing the TOP2A index are correlated, but that the inclusion of more than one gene improved the performance of a test based on this index.

When they evaluated the predictive performance of this index in subgroups of patients defined by the FISH evaluation of HER2, they observed a highly significant C-index of 0.84 (95% CI: 0.71-0.97, p=10⁻⁷) in the HER2+ subgroup, whereas it was not significant in the HER2− subgroup [0.47 (95% CI: 0.32-0.63), p=0.37)].

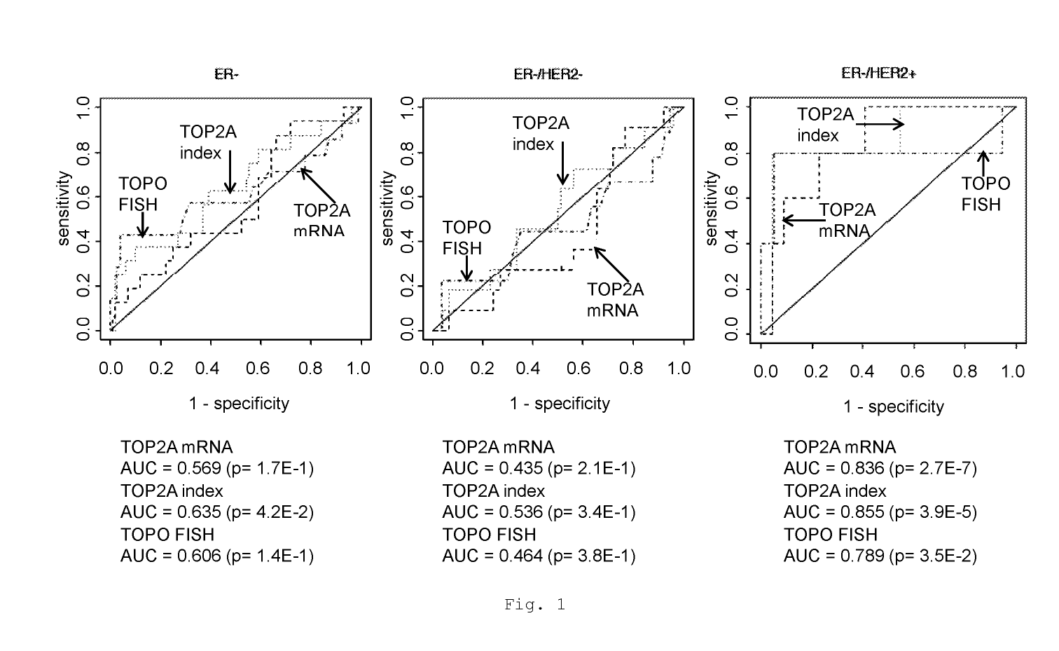

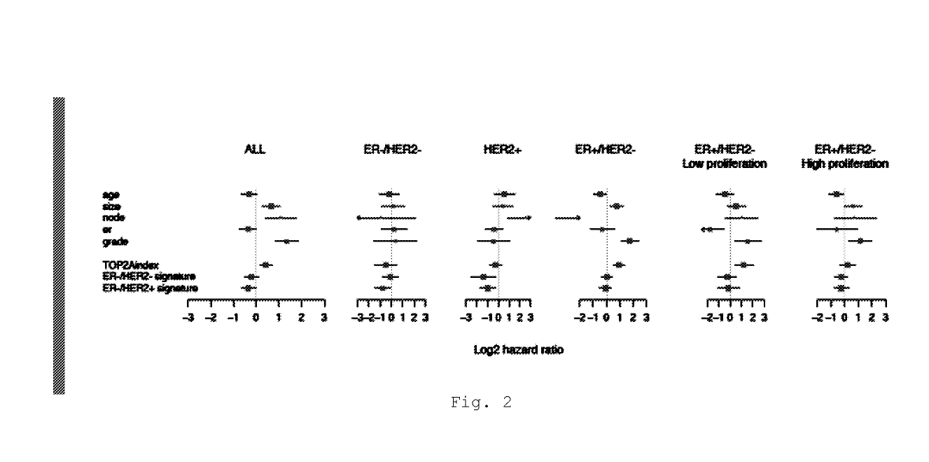

The ROC curves of the TOP2A gene expression and TOP2A index and their corresponding AUC, which can be interpreted as the C-index, are illustrated in FIG. 1 for both patient cohorts. The investigation of the prognostic value of the TOP2A index in untreated breast cancer patients using publicly available gene expression data showed that this index was not prognostic in the ER−/HER2− and HER2+ patients, but that high values of this index were associated with worse prognosis in the ER+/HER2 patients (FIG. 2). More precisely, this signature was associated with prognosis in the low proliferative (“luminal A”) subgroup of ER+/HER2− patients but not in the high proliferative subgroup (“luminal B”), suggesting that this index is not simply a good quantification of proliferation.

The inventors further validated the gene index they developed using the publicly available data of the EORTC 10994/BIG00-01 clinical trial (Bonnefoi et al., 2007, Lancet Oncology, 8, 1071-1078).

The inventors evaluated the predictive performance of this index in a cohort of patients treated pre-operatively either by an anthracyclines-based regimen (FEC) or by a taxane-based regimen (TET). It yielded a statistically significant C-index in the anthracyclines-arm [C-index=0.67 (95% CI: 0.54-0.79), p=0.006], but not in the taxane-based treatment arm [C-index=0.47 (95% CI: 0.32-0.62), p=0.35].

When studying the TOP2A index according to subgroups defined by HER2, the C-index was only predictive of response in the HER2+ [C-index=0.81 (95% CI: 0.63-1.00), p=0.0005] but not HER2− patients [C-index=0.56 (95% CI: 0.40-0.72), p=0.24] of the anthracyclines-arm. Of note, the TOP2A mRNA levels alone were not significantly associated with response in this validation cohort.

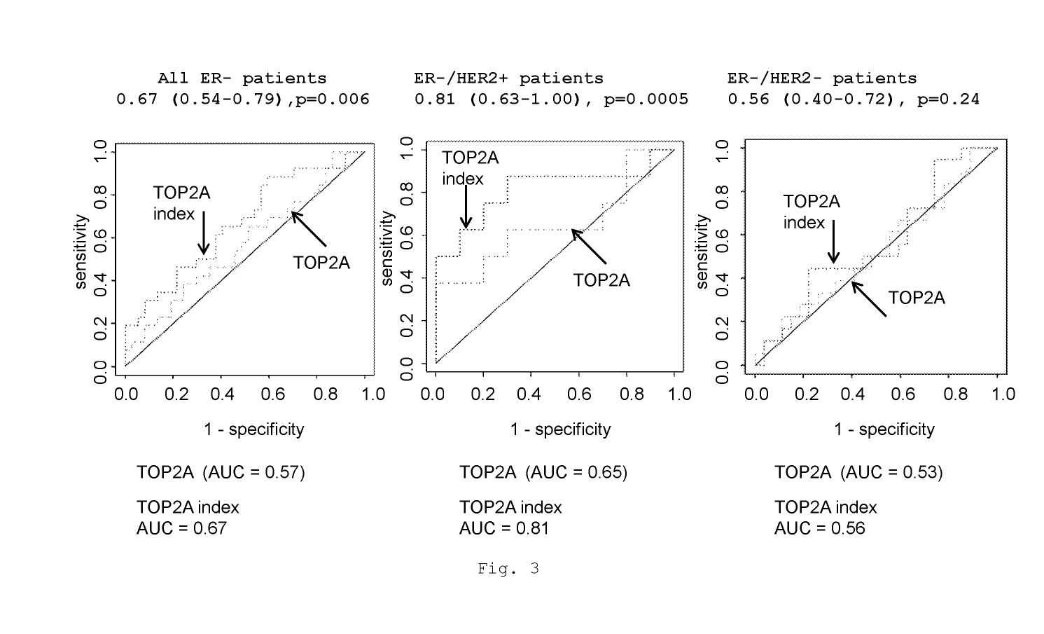

Again the inventors observed that TOP2A gene index predicts a better response to anthracyclines in the whole ER− patients and especially in ER−/HER2+ patients). Moreover, in this subsequent validation study, the TOP2A index clearly outperformed mRNA measurements of TOP2A amplification. (FIG. 3).

### Predictive Value of Biology-Driven Gene Expression Signatures

The inventors further investigated the predictive value of the gene expression modules that they developed previously in WO 2009/030770 and Desmedt et al. Clin Can Res 2008). In the global population, high levels of the estrogen receptor module were statistically associated with response to anthracyclines, whereas high levels of the tumor invasion module were associated with the presence of residual disease.

When looking into the ER−/HER− and ER−/HER2+ subgroups, the inventors observed that high levels of the tumor invasion module were associated with the presence of residual disease [C-index=0.31 (95% CI: 0.15-0.47), p=0.01 and 0.35 (95% CI: 0.19-0.50), p=0.02 respectively] in both subgroups.

Additionally, in the ER−/HER2+ subgroup, high values of the immune response module were associated with response to anthracyclines [C-index=0.75 (95% CI: 0.45-1.00), p=0.05]. Also the inventors have found that high values of the immune response module were associated with increased response rates to other chemotherapies and anti-cancer treatments.

TABLE 5
ER−/HER2− gene signature

| Gb acc. # | Symbol | Coef | Ugcluster | Gb acc. # | Symbol | Coef | Ugcluster | Gb acc. # | Symbol | Coef | Ugcluster |
|---|---|---|---|---|---|---|---|---|---|---|---|
| AU144449 | | −1 | Hs.469287 | NM_006419 | CXCL13 | 1 | Hs.100431 | NM_000213 | ITGB4 | −1 | Hs.370255 |
| BE550153 | | −1 | | NM_002416 | CXCL9 | 1 | Hs.77367 | AL049988 | ITPR2 | −1 | Hs.512235 |
| AL833150 | | 1 | Hs.327631 | AF361370 | CYB5R3 | −1 | Hs.517666 | NM_002223 | ITPR2 | −1 | Hs.512235 |
| AI087792 | | −1 | | AF003114 | CYR61 | −1 | Hs.8867 | NM_006801 | KDELR1 | −1 | Hs.515515 |
| AI192452 | | −1 | Hs.167793 | NM_006182 | DDR2 | −1 | Hs.275757 | AB033097 | KIAA1271 | −1 | Hs.567292 |
| BG389789 | | 1 | | AW571709 | DDX11 | −1 | Hs.443960 | BC002710 | KLK10 | −1 | Hs.275464 |
| AU155091 | | −1 | | NM_030881 | DDX17 | −1 | Hs.528305 | NM_006853 | KLK11 | −1 | Hs.57771 |
| R55784 | | −1 | Hs.155736 | U59321 | DDX17 | −1 | Hs.528305 | U90269 | KRIT1 | −1 | Hs.531987 |
| BF056892 | | −1 | Hs.435132 | NM_001356 | DDX3X | −1 | Hs.380774 | AJ278245 | LANCL2 | −1 | Hs.224282 |
| BF433269 | | −1 | Hs.208690 | NM_015362 | DERP6 | −1 | Hs.417029 | BE222220 | LEMD1 | −1 | Hs.181245 |
| AK024243 | | −1 | Hs.557780 | NM_012242 | DKK1 | −1 | Hs.40499 | NM_002305 | LGALS1 | −1 | Hs.445351 |
| AW392551 | | 1 | Hs.180559 | AF176013 | DNAJC12 | −1 | Hs.260720 | BF345728 | LOC14772 | −1 | Hs.534560 |
| BF969544 | | −1 | Hs.523913 | AL119957 | DNAJC3 | −1 | Hs.59214 | BM666010 | LOC20016 | −1 | |
| AA903473 | | 1 | Hs.245545 | AI692645 | DOCK5 | −1 | Hs.195403 | BF028225 | LOC20189 | −1 | Hs.205952 |
| AF495383 | ADAM9 | −1 | Hs.2442 | AL049369 | DSCR1 | −1 | Hs.282326 | AF519622 | LOC28302 | 1 | Hs.255729 |
| S68290 | AKR1C1 | −1 | Hs.558319 | NM_001943 | DSG2 | −1 | Hs.412597 | AI285192 | LOC28488 | 1 | |
| U05598 | AKR1C2 | −1 | | NM_004950 | DSPG3 | −1 | Hs.435680 | AV721528 | LOC28616 | −1 | |
| M33376 | AKR1C2 | −1 | | BC003143 | DUSP6 | −1 | Hs.298654 | BF970340 | LOC33944 | −1 | Hs.103939 |
| NM_001629 | ALOX5AP | −1 | Hs.507658 | AW269645 | ECT2 | 1 | Hs.518299 | BF211019 | LOC40094 | −1 | Hs.502948 |
| BE670056 | ANKRD10 | −1 | Hs.525163 | AA534817 | EDG3 | −1 | | AK021807 | LRP11 | −1 | Hs.408355 |
| AI097229 | ANKRD22 | 1 | Hs.217484 | S81545 | EDNRA | −1 | Hs.183713 | AB017498 | LRP5 | −1 | Hs.6347 |
| NM_018685 | ANLN | −1 | Hs.62180 | NM_015507 | EGFL6 | 1 | Hs.12844 | AF196468 | LSM2 | −1 | Hs.103106 |
| NM_006305 | ANP32A | −1 | Hs.458747 | U48722 | EGFR | −1 | Hs.488293 | AA112507 | LSM4 | −1 | Hs.515255 |
| NM_018153 | ANTXR1 | −1 | Hs.165859 | U95089 | EGFR | −1 | Hs.488293 | BE566894 | LTB4DH | −1 | Hs.555920 |
| N48299 | APCDD1 | −1 | Hs.293274 | NM_005228 | EGFR | −1 | Hs.488293 | NM_002343 | LTF | 1 | Hs.565960 |
| AY113699 | APH1A | −1 | Hs.108408 | AK000106 | EGFR | −1 | Hs.488293 | W80468 | MALAT1 | 1 | Hs.187199 |
| NM_017855 | APIN | −1 | Hs.143811 | NM_005228 | EGFR | −1 | Hs.488293 | AI936566 | MCM4 | −1 | Hs.460184 |
| BG427393 | APLP2 | −1 | Hs.370247 | BE878463 | EGFR | −1 | Hs.488293 | BC007207 | MGC1311 [symbol] | −1 | Hs.239500 |
| NM_001647 | APOD | −1 | Hs.522555 | NM_001415 | EIF2S3 | −1 | Hs.539684 | H09657 | MGC3990 [symbol] | 1 | Hs.496530 |
| AI358867 | APOE | 1 | Hs.515465 | U88968 | ENO1 | −1 | Hs.517145 | BF677486 | MGC3990 [symbol] | 1 | Hs.496530 |
| N74607 | AQP3 | 1 | Hs.234642 | BC001038 | EPN3 | 1 | Hs.165904 | AK026366 | MGC4862 [symbol] | −1 | Hs.558780 |
| AF052179 | ARF1 | −1 | Hs.286221 | BG479856 | FAM60A | −1 | Hs.505154 | BE855713 | MGC9913 | −1 | Hs.23133 |
| AI343000 | ARGBP2 | −1 | Hs.481342 | NM_006329 | FBLN5 | −1 | Hs.332708 | NM_006533 | MIA | −1 | Hs.279651 |
| BC040474 | ARHGEF1 | −1 | Hs.98594 | NM_002006 | FGF2 | −1 | Hs.284244 | NM_002427 | MMP13 | −1 | Hs.2936 |
| AF019888 | ARPC4 | −1 | Hs.323342 | NM_002014 | FKBP4 | −1 | Hs.524183 | N74662 | MRPL43 | 1 | Hs.421848 |
| NM_004318 | ASPH | −1 | Hs.332422 | BI087313 | FLJ10707 | −1 | Hs.288164 | AB049959 | MRPL51 | −1 | Hs.55847 |
| AF306765 | ASPH | −1 | Hs.332422 | AK075525 | FLJ14712 | −1 | Hs.50802 | AK022122 | MTBP | −1 | Hs.553528 |
| D13119 | ATP5G2 | −1 | Hs.524464 | NM_144682 | FLJ31952 | −1 | Hs.462833 | NM_006097 | MYL9 | −1 | Hs.504687 |
| NM_001690 | ATP6V1A | −1 | Hs.477155 | AA507012 | FLOT1 | −1 | Hs.179986 | W74452 | NDRG2 | −1 | Hs.525205 |
| AB028869 | BIRC5 | −1 | Hs.514527 | AJ276395 | FN1 | −1 | Hs.203717 | BE741920 | NDUFA11 | −1 | Hs.406062 |
| NM_001200 | BMP2 | −1 | Hs.73853 | NM_016725 | FOLR1 | −1 | Hs.73769 | AI357376 | NEDD4L | −1 | Hs.185677 |
| L20471 | BSG | −1 | Hs.501293 | NM_005249 | FOXG1B | −1 | Hs.525266 | AA005023 | NOD27 | 1 | Hs.528836 |
| AL049332 | BTG3 | −1 | Hs.473420 | BC004908 | FSCN1 | −1 | Hs.118400 | AA694067 | NOD3 | 1 | Hs.128357 |
| AW204712 | C10orf128 | 1 | Hs.385493 | BE930017 | FUS | −1 | Hs.513522 | AI868441 | NPW | −1 | Hs.233533 |
| AC007182 | C14orf1 | −1 | Hs.15106 | AF072872 | FZD1 | −1 | Hs.94234 | NM_014581 | OBP2B /// | −1 | Hs.449629 |
| BC007010 | C1S | −1 | Hs.458355 | AI796169 | GATA3 | 1 | Hs.524134 | NM_003999 | OSMR | −1 | Hs.120658 |
| NM_014145 | C20orf30 | −1 | Hs.472024 | AI796169 | GATA3 | 1 | Hs.524134 | AI569974 | OSR1 | −1 | Hs.123933 |
| BF381837 | C20orf52 | −1 | Hs.472564 | NM_002053 | GBP1 | 1 | Hs.62661 | AK075503 | P4HB | −1 | Hs.464336 |
| AW001030 | C9orf52 | 1 | Hs.130086 | AL136680 | GBP3 | 1 | Hs.534284 | AI608902 | PDCD1LG | 1 | Hs.521989 |
| AF257659 | CALU | −1 | Hs.7753 | BG271923 | GBP5 | 1 | Hs.513726 | U67932 | PDE7A | −1 | Hs.527119 |
| NM_004058 | CAPS | −1 | Hs.567229 | NM_019067 | GNL3L | −1 | Hs.522664 | AB033831 | PDGFC | −1 | Hs.148162 |
| L07555 | CD69 | 1 | Hs.208854 | AF064826 | GPC4 | −1 | Hs.58367 | BG054550 | PDLIM5 | −1 | Hs.480311 |
| AW006735 | CD8A | 1 | Hs.85258 | NM_013308 | GPR171 | 1 | Hs.549152 | AF116705 | PDLIM5 | −1 | Hs.480311 |
| BC003682 | CDC42 | −1 | Hs.467637 | U87460 | GPR37 | 1 | Hs.406094 | NM_000287 | PEX6 | −1 | Hs.567243 |
| NM_004360 | CDH1 | −1 | Hs.461086 | NM_005756 | GPR64 | −1 | Hs.146978 | NM_002624 | PFDN5 | −1 | Hs.288856 |
| D21254 | CDH11 | −1 | Hs.116471 | NM_000826 | GRIA2 | −1 | Hs.32763 | NM_016134 | PGCP | −1 | Hs.156178 |
| BC015877 | CDH19 | −1 | Hs.42771 | NM_000846 | GSTA1 | −1 | Hs.446309 | R81072 | PHACTR2 | −1 | Hs.102471 |
| AA922068 | CDK6 | −1 | Hs.119882 | BF569051 | H19 | −1 | Hs.556040 | AV721177 | PICALM | −1 | Hs.163893 |
| AW192700 | CDK6 | −1 | Hs.119882 | NM_017445 | H2BFS | −1 | Hs.473961 | NM_002645 | PIK3C2A | −1 | Hs.175343 |
| D00682 | CFL1 | −1 | Hs.170622 | NM_007071 | HHLA3 | −1 | Hs.142245 | NM_003628 | PKP4 | −1 | Hs.407580 |
| AK025141 | CHPT1 | −1 | Hs.293077 | AC007130 | HIBADH | −1 | Hs.406758 | K03226 | PLAU | −1 | Hs.77274 |
| AF279779 | CHRM3 | −1 | Hs.7138 | NM_152696 | HIPK1 | −1 | Hs.532363 | Z54367 | PLEC1 | −1 | Hs.434248 |
| T15991 | CHRM3 | −1 | Hs.7138 | BC002649 | HIST1H1C | −1 | Hs.7644 | AW469573 | PLEKHC1 | −1 | Hs.509343 |
| AK056349 | CHRM3 | −1 | Hs.559103 | NM_003514 | HIST1H2A | −1 | Hs.134999 | AI754404 | PLOD2 | −1 | Hs.477866 |
| NM_000740 | CHRM3 | −1 | Hs.7138 | NM_003523 | HIST1H2B | −1 | Hs.534369 | AW665155 | POLH | −1 | Hs.439153 |
| NM_006984 | CLDN10 | −1 | Hs.534377 | NM_003522 | HIST1H2B | −1 | Hs.182137 | BQ613856 | POLR2J2 | −1 | Hs.533383 |
| N74924 | CLDN20 | −1 | Hs.352244 | BE271470 | HIST1H2B | −1 | Hs.352109 | BC005903 | POLR2L | −1 | Hs.441072 |
| BE791251 | CLDN3 | −1 | Hs.25640 | NM_003525 | HIST1H2B | −1 | Hs.553506 | NM_000942 | PPIB | −1 | Hs.434937 |
| Y15916 | COL1A1 | −1 | Hs.172928 | AI313324 | HIST2H2A | −1 | Hs.530461 | AB041836 | PQBP1 | −1 | Hs.534384 |
| AA909035 | COL4A2 | −1 | Hs.508716 | BC001629 | HIST2H2A | −1 | Hs.530461 | BC017833 | PRIM2A | −1 | Hs.485640 |
| M20776 | COL6A1 | −1 | Hs.474053 | U70544 | HLA-DRB4 | −1 | Hs.534321 | AL121975 | PRIM2A | −1 | Hs.485640 |
| AY029208 | COL6A2 | −1 | Hs.420269 | NM_002130 | HMGCS1 | −1 | Hs.397729 | NM_014086 | PRO1073 | 1 | |
| NM_052889 | COP1 | 1 | Hs.348365 | BF983406 | HNRPH1 | −1 | Hs.202166 | NM_144707 | PROM2 | 1 | Hs.469313 |
| AI621079 | COPA | −1 | | NM_020386 | HRASLS | 1 | Hs.36761 | AI184802 | PRPF4 | −1 | Hs.374973 |
| NM_001863 | COX6B1 | −1 | Hs.431668 | BG612458 | HSPCB | −1 | Hs.509736 | NM_007244 | PRR4 | −1 | Hs.408153 |
| AF142573 | CRISPLD1 | −1 | Hs.436542 | D13889 | ID1 | −1 | Hs.504609 | BF576710 | PTP4A1 | −1 | Hs.227777 |
| BC008745 | CRTAP | −1 | Hs.517888 | BC001872 | IGHM | 1 | Hs.525648 | AU147115 | PTPRK | −1 | Hs.155919 |
| NM_005212 | CSN3 | −1 | Hs.54415 | AU160004 | IMP-3 | −1 | Hs.432616 | AF312393 | PTRF | −1 | Hs.437191 |
| BG330076 | CTNNA1 | −1 | Hs.445981 | AV733308 | ITGA6 | −1 | Hs.133397 | NM_016277 | RAB23 | −1 | Hs.555016 |
| AY072911 | CXADR | −1 | Hs.473417 | NM_033669 | ITGB1 | −1 | Hs.429052 | AB029004 | RAB6IP2 | −1 | Hs.400431 |
| NM_001565 | CXCL10 | 1 | Hs.413924 | BF305661 | ITGB4 | −1 | Hs.370255 | NM_006506 | RASA2 | −1 | Hs.15999 |
| AF493929 | RGS5 | −1 | Hs.24950 | AL121985 | SLAMF7 | 1 | Hs.517265 | K03199 | TP53 | −1 | Hs.408312 |
| AF498970 | RHOA | −1 | Hs.247077 | BG251467 | SLC25A37 | −1 | Hs.122514 | AW665624 | TPARL | −1 | Hs.479766 |
| BI668074 | RHOB | −1 | Hs.502876 | NM_004955 | SLC29A1 | −1 | Hs.25450 | BG389015 | TPD52 | −1 | Hs.368433 |
| BC025770 | RHOJ | −1 | Hs.525389 | BC039498 | SLC39A6 | −1 | Hs.79136 | NM_000366 | TPM1 | −1 | Hs.133892 |
| NM_002933 | RNASE1 | −1 | Hs.78224 | AL137517 | SLITRK6 | −1 | Hs.525105 | AA602532 | TPP1 | −1 | Hs.523454 |
| NM_000980 | RPL18A // | −1 | Hs.558383 | NM_003064 | SLPI | −1 | Hs.517070 | AW235355 | TPR | −1 | Hs.279640 |
| NM_000978 | RPL23 | −1 | Hs.406300 | AK026426 | SMARCA1 | −1 | Hs.152292 | NM_003294 | TPSAB1 | −1 | |
| NM_000991 | RPL28 | −1 | Hs.356371 | AI989477 | SOX4 | −1 | Hs.357901 | AF206666 | TPSAB1 | −1 | |
| NM_001004 | RPLP2 | −1 | Hs.437594 | BC002704 | STAT1 | 1 | Hs.470943 | AF099143 | TPSAB1 | −1 | |
| NM_001020 | RPS16 | −1 | Hs.397609 | U46768 | STC1 | −1 | Hs.25590 | AF206667 | TPSAB1 // | −1 | Hs.405479 |
| NM_012250 | RRAS2 | −1 | Hs.502004 | AL553320 | STIP1 | −1 | Hs.337295 | NM_003293 | TPSAB1 // | −1 | Hs.405479 |
| AI692974 | RRM1 | −1 | Hs.558393 | NM_007271 | STK38 | −1 | Hs.409578 | NM_024164 | TPSB2 | −1 | Hs.405479 |
| AK022166 | RSBN1 | −1 | Hs.486285 | AF008937 | STX16 | −1 | Hs.307913 | BI857154 | TRAM1 | −1 | Hs.491988 |
| NM_006054 | RTN3 | −1 | Hs.473761 | AL034418 | SULF2 | −1 | Hs.162016 | AI884858 | TUSC3 | −1 | Hs.426324 |
| NM_006271 | S100A1 | −1 | Hs.515715 | U46837 | SURB7 | −1 | Hs.286145 | U42349 | TUSC3 | −1 | Hs.426324 |
| AW238654 | S100A8 | 1 | Hs.416073 | AF077053 | TAF9L | −1 | Hs.546635 | NM_016267 | VGLL1 | −1 | Hs.496843 |
| BC001766 | S100B | −1 | Hs.422181 | BC010946 | TAGLN | −1 | Hs.503998 | BE542323 | VGLL1 | −1 | Hs.496843 |
| NM_007281 | SCRG1 | −1 | Hs.7122 | BG330520 | TALDO1 // | −1 | Hs.356766 | AF199015 | VIL2 | −1 | Hs.487027 |
| AI380298 | SDC2 | −1 | Hs.1501 | NM_003220 | TFAP2A | −1 | Hs.519880 | BF663141 | VIL2 | −1 | Hs.487027 |
| AI380298 | SDC2 | −1 | Hs.1501 | AF047002 | THOC4 | −1 | Hs.534385 | NM_016312 | WBP11 | −1 | Hs.524281 |
| AW294630 | SEC15L2 | −1 | Hs.303454 | U67195 | TIMP3 | −1 | Hs.297324 | AF274954 | WDR1 | −1 | Hs.128548 |
| AF346602 | SEC61A1 | −1 | Hs.518236 | BC005176 | TM7SF3 | −1 | Hs.438641 | AI445745 | WNK1 | −1 | Hs.504432 |
| AL574210 | SERPINE [symbol] | −1 | Hs.414795 | NM_016551 | TM7SF3 | −1 | Hs.438641 | AF303378 | YSG2 | −1 | Hs.10056 |
| Z95126 | SET /// LO | −1 | Hs.436687 | BF540749 | TM7SF3 | −1 | Hs.438641 | BC003623 | YWHAZ | −1 | Hs.558651 |
| AF131749 | SEZ6L2 | −1 | Hs.6314 | AB004064 | TMEFF2 | 1 | Hs.144513 | BG483802 | ZBTB10 | −1 | Hs.205742 |
| NM_006275 | SFRS6 | −1 | Hs.6891 | BE568134 | TNFRSF21 | −1 | Hs.443577 | NM_016620 | ZNF644 | −1 | Hs.173001 |

[symbol]

indicates data missing or illegible when filed

### De Novo Identification of Predictive Gene Expression Signatures

Given the unique characteristics of the TOP trial, i.e. that patients were treated with anthracyclines monotherapy in the neo-adjuvant setting; the inventors aimed at developing de novo gene expression signatures that would predict the efficacy of anthracyclines both in ER−/HER2− and ER−/HER2+ tumors (Tables 5 and 6).

The ER−/HER2− gene signature was composed of 321 probe-sets corresponding to 294 unique genes (Table 6). The signature included PLAU, which is the prototype from the tumor invasion gene expression module of WO 2009/030770 and of Table 13 of the present invention; EGFR whose over-expression has been correlated with poor survival (Bartlett et al. 2008) and with resistance to anthracyclines-based chemotherapy in triple-negative breast cancer, TP53 whose predictive value for efficacy of anthracyclines-based chemotherapy has been matter of controversy and many others.

The IAP analysis, which included 218 genes, revealed that cellular movement, cellular growth and proliferation, cell-to-cell signaling, cell death and cellular assembly were the most significant functional classes.

The ER−/HER2+ gene signature was composed of 261 probe-sets corresponding to 218 unique genes. Eleven genes were in common with the ER−/HER2+ signature and both signatures displayed a low but significant correlation (p=0.22, p=0.02). As expected, this signature included TOP2A as well as other genes located on 17q21-q22. Of interest, IGF1R, GRB7, a gene located close to HER2, and different metallothioneins were also part of this signature and their over-expression was associated with the presence of residual disease.

TABLE 6
ER−/HER2+ gene signature

| Gb acc. # | Symbol | Coef | Ugcluster | Gb acc. # | Symbol | Coef | Ugcluster | Gb acc. # | Symbol | Coef | Ugcluster |
|---|---|---|---|---|---|---|---|---|---|---|---|
| AW574798 | KLHL6 | 1 | Hs.333181 | M24669 | IGHM | 1 | Hs.525648 | NM_014258 | SYCP2 | −1 | Hs.202676 |
| AW043921 | | −1 | Hs.547618 | M87789 | IGH@ /// I [symbol] | 1 | Hs.567449 | AA770014 | DSCR8 | 1 | Hs.192371 |
| AF103530 | | 1 | Hs.559330 | L14454 | IGHG3 | 1 | Hs.510635 | AB014341 | IGLJ3 | 1 | Hs.517453 |
| X93006 | | 1 | Hs.561078 | AJ275439 | IGHA1 /// [symbol] | 1 | Hs.497723 | AB001733 | IGLJ3 | 1 | Hs.517453 |
| AF043583 | | 1 | Hs.449599 | U80139 | IGHM | 1 | Hs.525648 | X57812 | IGL@ /// IGL | 1 | Hs.449585 |
| AJ239383 | | 1 | Hs.551925 | AJ243643 | IGHA1 /// [symbol] | 1 | Hs.497723 | AA680302 | IGL@ /// IGL | 1 | Hs.449585 |
| X84340 | | 1 | | AJ275408 | IGHA1 /// I [symbol] | 1 | Hs.497723 | AV698647 | IGL@ /// IGL | 1 | Hs.449585 |
| AL359605 | | 1 | Hs.283851 | U80164 | IGH@ /// I [symbol] | 1 | Hs.567449 | D84143 | IGLC2 | 1 | Hs.567242 |
| AF043584 | | 1 | Hs.449599 | BF002659 | IGHM | 1 | Hs.525648 | D87021 | IGLC2 | 1 | Hs.567242 |
| AI743780 | | −1 | | BC001872 | IGHM | 1 | Hs.525648 | AF103591 | IGLC2 | 1 | Hs.567242 |
| AI739132 | | −1 | Hs.152812 | NM_01479 | KIAA0125 | 1 | Hs.395486 | AJ249377 | IGLC2 | 1 | Hs.567242 |
| AI923633 | | 1 | Hs.96886 | S55735 | IGHA1 /// I [symbol] | 1 | Hs.558339 | NM_017424 | CECR1 | 1 | Hs.170310 |
| BF476080 | | 1 | Hs.418040 | AJ275355 | IGHG1 /// [symbol] | 1 | Hs.497723 | AF043586 | IGLC1 | 1 | Hs.555877 |
| AK025909 | | −1 | Hs.288741 | X17115 | IGHM | 1 | Hs.525648 | M87790 | IGLC2 | 1 | Hs.567242 |
| BF108778 | | 1 | Hs.28360 | AJ275397 | IGHG1 /// I [symbol] | 1 | Hs.497723 | D01059 | IGLC2 | 1 | Hs.567242 |
| AI004009 | | −1 | Hs.130526 | AB035175 | IGHA1 /// I [symbol] | 1 | Hs.525648 | U96394 | IGLC2 | 1 | Hs.449585 |
| L06102 | | 1 | Hs.547404 | U92706 | IGHA1 /// I [symbol] | 1 | Hs.510635 | L21961 | IGLC2 | 1 | Hs.567242 |
| NM_052889 | COP1 | 1 | Hs.348365 | AJ225092 | IGHA1 /// I [symbol] | 1 | Hs.510635 | D84140 | IGLC2 | 1 | Hs.567242 |
| AL833150 | | −1 | Hs.327631 | BG340548 | IGH@ /// I [symbol] | 1 | Hs.558339 | H53689 | IGLV3-25 | 1 | |
| AK097976 | KLHL6 | 1 | Hs.333181 | NM_00302 | SGNE1 | −1 | Hs.156540 | AJ249377 | IGLV2-14 | 1 | |
| M74303 | | 1 | Hs.560200 | NM_02014 | MEIS2 | −1 | Hs.510989 | D87016 | IGLC2 | 1 | Hs.449567 |
| L23516 | | 1 | Hs.383169 | BC003610 | MFGE8 | −1 | Hs.3745 | AI761713 | MMP11 | −1 | Hs.143751 |
| AF103529 | | 1 | Hs.560823 | NM_00257 | PCSK6 | −1 | Hs.498494 | AI655697 | DERL3 | 1 | Hs.159971 |
| L06101 | | 1 | Hs.64568 | BC008777 | ITGAL | 1 | Hs.174103 | AL022324 | LOC91353 | 1 | Hs.546463 |
| BG536224 | | 1 | Hs.559333 | BC025741 | C16orf54 | 1 | Hs.331095 | AA398569 | LOC91316 | 1 | Hs.148656 |
| M85256 | | 1 | Hs.554197 | AI377875 | EIF3S8 | −1 | Hs.192425 | NM_005080 | XBP1 | 1 | Hs.437638 |
| L34164 | | 1 | Hs.448957 | AA102581 | USP31 | −1 | Hs.183817 | AL008583 | NPTXR | −1 | Hs.91622 |
| M87268 | | 1 | Hs.448957 | NM_00584 | IGSF6 | 1 | Hs.530902 | NM_021822 | APOBEC3G | 1 | Hs.474853 |
| BG482805 | | 1 | Hs.551722 | NM_00119 | TNFRSF17 | 1 | Hs.2556 | NM_025225 | ADPN | −1 | Hs.377087 |
| AW404894 | | 1 | Hs.552522 | AI401105 | TXNDC11 | 1 | Hs.313847 | AK025665 | ADPN | −1 | Hs.377087 |
| AW405975 | | 1 | Hs.449575 | U90304 | IRX5 | −1 | Hs.435730 | AI631846 | MGC16635 | 1 | Hs.137007 |
| L23518 | | 1 | Hs.560223 | M10943 | MT1F | −1 | Hs.513626 | NM_000878 | IL2RB | 1 | Hs.474787 |
| BE620374 | | −1 | Hs.559452 | BF217861 | MT1E | −1 | Hs.534330 | M20812 | LOC339562 | 1 | Hs.449972 |
| AI225238 | | 1 | Hs.445500 | NM_00595 | MT1X | −1 | Hs.374950 | BG540628 | IGKV1-5 | 1 | |
| BF244402 | | −1 | | NM_02471 | ELMO3 | −1 | Hs.377416 | AJ408433 | IGKC | 1 | Hs.449621 |
| BF244402 | | −1 | | NM_00174 | CALB2 | −1 | Hs.106857 | M63438 | IGKC /// IGK | 1 | Hs.449621 |
| AI803010 | | −1 | Hs.554052 | NM_02430 | FA2H | 1 | Hs.461329 | BC005332 | IGKC /// IGK | 1 | Hs.449621 |
| AW628735 | | 1 | Hs.567989 | NM_01485 | KIAA0672 | 1 | Hs.499758 | L14457 | IGKC | 1 | Hs.449621 |
| AW504569 | | 1 | Hs.551955 | BC005926 | EVI2B | 1 | Hs.5509 | AW408194 | IGKV1D-13 | 1 | Hs.390427 |
| AI660245 | | 1 | Hs.547730 | AB000221 | CCL18 | 1 | Hs.143961 | L14458 | IGKC | 1 | Hs.449621 |
| AA424537 | C10orf18 | −1 | Hs.432548 | Y13710 | CCL18 | 1 | Hs.143961 | BG548679 | IGKC | 1 | Hs.449621 |
| NM_000698 | ALOX5 | 1 | Hs.89499 | NM_00735 | CASC3 | 1 | Hs.350229 | AW575927 | IGKC /// IGK | 1 | Hs.449621 |
| AL118571 | C10orf74 | −1 | Hs.499833 | AF061812 | KRT16 | −1 | Hs.432448 | AW006735 | CD8A | 1 | Hs.85258 |
| U11058 | KCNMA1 | 1 | Hs.144795 | AA001203 | LOC33928 | 1 | Hs.532786 | BG485135 | IGKC | 1 | Hs.449621 |
| AI917901 | ACTA2 | −1 | Hs.500483 | AI702962 | SMARCE1 | 1 | Hs.463010 | M85276 | GNLY | 1 | Hs.105806 |
| BE888744 | IFIT2 | −1 | Hs.437609 | NM_00125 | CDC6 | 1 | Hs.405958 | NM_006433 | GNLY | 1 | Hs.105806 |
| AW043782 | LDLRAD3 | −1 | Hs.205865 | U77949 | CDC6 | 1 | Hs.405958 | NM_001615 | ACTG2 | −1 | Hs.516105 |
| AI755024 | LOC38775 | −1 | Hs.32478 | AA046439 | SFRS1 | 1 | Hs.68714 | AK022277 | DTNB | 1 | Hs.307720 |
| NM_030754 | SAA1 /// S | −1 | Hs.332053 | AU159942 | TOP2A | 1 | Hs.156346 | BE465475 | KBTBD9 | −1 | Hs.130593 |
| AL042088 | TUB | −1 | Hs.231850 | AL561834 | TOP2A | 1 | Hs.156346 | AI337069 | RSAD2 | −1 | Hs.17518 |
| AI928342 | ASRGL1 | 1 | Hs.535326 | AK000271 | SUPT4H1 | 1 | Hs.439481 | AW004016 | ST6GAL2 | −1 | Hs.98265 |
| NM_025080 | ASRGL1 | 1 | Hs.535326 | AI082827 | MPPE1 | −1 | Hs.514713 | NM_001450 | FHL2 | −1 | Hs.443687 |
| NM_000852 | GSTP1 | 1 | Hs.523836 | NM_02112 | PMAIP1 | −1 | Hs.96 | X51887 | LOC391427 | 1 | |
| U94592 | UCP2 | 1 | Hs.80658 | NM_02112 | PMAIP1 | −1 | Hs.96 | AI928242 | TFCP2L1 | −1 | Hs.156471 |
| AA725246 | SPTBN2 | 1 | Hs.26915 | NM_00343 | ZNF91 | 1 | Hs.558418 | AF255647 | DKFZP566N | 1 | Hs.369471 |
| AI246687 | CTSC | 1 | Hs.128065 | NM_00164 | APOC1 | 1 | Hs.110675 | AU146532 | PDK1 | 1 | Hs.470633 |
| AI246687 | CTSC | 1 | Hs.128065 | NM_01211 | CBLC | −1 | Hs.466907 | BF060783 | DLX1 | −1 | Hs.407015 |
| NM_002421 | MMP1 | 1 | Hs.83169 | NM_00277 | KLK6 | −1 | Hs.79361 | NM_000648 | CCR2 | 1 | Hs.511794 |
| NM_019604 | CRTAM | 1 | Hs.159523 | AF243527 | KLK5 | −1 | Hs.50915 | AA225165 | LOC339903 | −1 | Hs.146346 |
| NM_006235 | POU2AF1 | 1 | Hs.2407 | NM_00719 | KLK8 | −1 | Hs.104570 | R42166 | CNTN4 | −1 | Hs.298705 |
| AF329841 | C1QTNF5 | −1 | Hs.157211 | AL042588 | PEG3 | −1 | Hs.201776 | AJ240085 | TRAT1 | 1 | Hs.138701 |
| BG112263 | ASAM | −1 | Hs.504187 | AF208967 | PEG3 | −1 | Hs.201776 | AW170015 | PLCXD2 | 1 | Hs.292419 |
| NM_003622 | PPFIBP1 | −1 | Hs.172445 | NM_00504 | KLK7 | −1 | Hs.151254 | NM_018456 | EAF2 | 1 | Hs.477325 |
| BC005961 | PTHLH | −1 | Hs.89626 | AL046017 | FAM46C | 1 | Hs.356216 | AF303889 | ROPN1 | −1 | Hs.558504 |
| M31157 | PTHLH | −1 | Hs.89626 | NM_00176 | CD2 | 1 | Hs.523500 | AI340264 | ROPN1 | −1 | Hs.558504 |
| NM_002258 | KLRB1 | 1 | Hs.169824 | AI743596 | TSPAN2 | −1 | Hs.310458 | NM_017578 | ROPN1B | −1 | Hs.528203 |
| NM_001038 | SCNN1A | 1 | Hs.130989 | AI082747 | PALMD | −1 | Hs.483993 | AI674183 | EPHB1 | −1 | Hs.116092 |
| NM_007360 | KLRK1 | 1 | Hs.387787 | NM_00260 | PDE4B | 1 | Hs.198072 | NM_000685 | AGTR1 | −1 | Hs.477887 |
| NM_001769 | CD9 | −1 | Hs.114286 | L20966 | PDE4B | 1 | Hs.198072 | AI754404 | PLOD2 | −1 | Hs.477866 |
| AY007436 | RBP5 | 1 | Hs.246046 | AI149963 | DKFZp761 | −1 | Hs.132121 | AI684991 | CP | 1 | Hs.554736 |
| NM_003979 | GPRC5A | −1 | Hs.194691 | AA456955 | ANKRD38 | −1 | Hs.283398 | NM_000096 | CP | 1 | Hs.554736 |
| AK026776 | LRRK2 | 1 | Hs.187636 | NM_01240 | PLA2G2D | 1 | Hs.189507 | AI922198 | HPS3 /// CP | 1 | Hs.477898 |
| NM_173600 | MUC19 | 1 | Hs.244017 | AW24198 [symbol] | FCRL5 | 1 | Hs.415950 | NM_022443 | MLF1 | −1 | Hs.85195 |
| J00269 | KRT6A /// | −1 | Hs.558758 | AF343662 | FCRL5 | 1 | Hs.415950 | AI911434 | MLF1 | −1 | Hs.85195 |
| U89281 | HSD17B6 | −1 | Hs.524513 | NM_00146 | FMO5 | 1 | Hs.303476 | NM_014398 | LAMP3 | 1 | Hs.518448 |
| NM_000785 | CYP27B1 | 1 | Hs.524528 | AI659418 | RCSD1 | 1 | Hs.493867 | NM_006548 | IMP-2 | −1 | Hs.35354 |
| BF510098 | PPP1R1A | 1 | Hs.505662 | AL121985 | SLAMF7 | 1 | Hs.517265 | AI743792 | ST6GAL1 | 1 | Hs.207459 |
| NM_001874 | CPM | 1 | Hs.484551 | AL121985 | SLAMF7 | 1 | Hs.517265 | AA702685 | OSTalpha | 1 | Hs.567320 |
| NM_004950 | DSPG3 | 1 | Hs.435680 | AJ271869 | SLAMF7 | 1 | Hs.517265 | AI126453 | COX7B2 | 1 | Hs.479656 |
| NM_013300 | HSU79274 | −1 | Hs.436618 | AL514445 | RGS4 | −1 | Hs.386726 | AK026815 | KIAA1102 | −1 | Hs.335163 |
| NM_007197 | FZD10 | −1 | Hs.31664 | NM_00065 | SELL | 1 | Hs.82848 | AI494113 | KIAA1102 | −1 | Hs.335163 |
| M86849 | GJB2 | −1 | Hs.524894 | M83772 | FMO3 | 1 | Hs.445350 | AI609256 | SLC30A9 | −1 | Hs.479634 |
| BF432648 | TNFRSF19 | −1 | Hs.149168 | AW003297 | RALGPS2 | 1 | Hs.496222 | AB018289 | KIAA0746 | 1 | Hs.479384 |
| AW137148 | POSTN | −1 | Hs.136348 | NM_00346 | CHIT1 | 1 | Hs.201688 | AA522514 | KIAA0746 | 1 | Hs.479384 |
| AL137517 | SLITRK6 | −1 | Hs.525105 | BG339064 | BTG2 | 1 | Hs.519162 | NM_002416 | CXCL9 | 1 | Hs.77367 |
| R70320 | SLITRK6 | −1 | Hs.525105 | NM_01777 | LAX1 | 1 | Hs.272794 | AV733266 | IGJ | 1 | Hs.381568 |
| AI680986 | SLITRK6 | −1 | Hs.525105 | BC014852 | IRF6 | −1 | Hs.355827 | AF116705 | PDLIM5 | −1 | Hs.480311 |
| AF193855 | ZIC2 | −1 | Hs.369063 | NM_00614 | IRF6 | −1 | Hs.355827 | BF671400 | PDLIM5 | −1 | Hs.480311 |
| AI057619 | UGCGL2 | −1 | Hs.193226 | M19154 | TGFB2 | −1 | Hs.133379 | AF065389 | TSPAN5 | 1 | Hs.118118 |
| M12959 | TRAC | 1 | | NM_00323 | TGFB2 | −1 | Hs.133379 | AA059445 | TSPAN5 | 1 | Hs.118118 |
| NM_004496 | FOXA1 | 1 | Hs.163484 | NM_00250 | NKX2-2 | −1 | Hs.516922 | NM_001977 | ENPEP | −1 | Hs.435765 |
| N79004 | SIX1 | 1 | Hs.558398 | NM_00311 | SPAG4 | 1 | Hs.123159 | NM_024090 | ELOVL6 | −1 | Hs.412939 |
| NM_000295 | SERPINA [symbol] | 1 | Hs.525557 | M35533 | LBP | 1 | Hs.154078 | BC001305 | ELOVL6 | −1 | Hs.412939 |
| L23519 | IGHV1-69 | 1 | Hs.449011 | AW024383 | RPS21 | −1 | Hs.190968 | BG545288 | FGB | 1 | Hs.300774 |
| M24670 | IGHV1-69 | 1 | Hs.449011 | NM_00195 | EEF1A2 | −1 | Hs.433839 | NM_005141 | FGB | 1 | Hs.300774 |
| NM_000509 | FGG | 1 | Hs.546255 | M32577 | HLA-DQB1 | 1 | Hs.409934 | NM_016027 | LACTB2 | −1 | Hs.118554 |
| NM_004362 | CLGN | 1 | Hs.86368 | NM_00352 | HIST1H2B [symbol] | −1 | Hs.546314 | BC040474 | ARHGEF10 | −1 | Hs.98594 |
| S81545 | EDNRA | −1 | Hs.183713 | AA037483 | HIST1H2B [symbol] | −1 | Hs.546314 | AF306765 | ASPH | 1 | Hs.332422 |
| NM_000909 | NPY1R | −1 | Hs.519057 | BC002842 | HIST1H2B [symbol] | −1 | Hs.130853 | NM_006823 | PKIA | 1 | Hs.433700 |
| NM_000163 | GHR | −1 | Hs.125180 | M16276 | HLA-DQB1 | −1 | Hs.409934 | BF245954 | PKIA | 1 | Hs.433700 |
| N71063 | ADAMTS6 | −1 | Hs.482291 | BE740761 | HIST1H4H | −1 | Hs.421737 | AL136588 | DKFZp761D | −1 | Hs.492187 |
| NM_017614 | BHMT2 | −1 | Hs.114172 | NM_00059 | C4A /// C4 | 1 | Hs.546241 | NM_012082 | ZFPM2 | −1 | Hs.431009 |
| AW206234 | FLJ42709 | −1 | Hs.171132 | NM_00354 | HIST1H4H | −1 | Hs.421737 | BF979497 | SQLE | −1 | Hs.71465 |
| BC030966 | KIAA0372 | −1 | Hs.482868 | M17955 | HLA-DQB1 | 1 | Hs.409934 | AL137725 | EPPK1 | 1 | Hs.200412 |
| NM_004772 | C5orf13 | −1 | Hs.483067 | NM_00322 | TFAP2B | 1 | Hs.33102 | AL137725 | EPPK1 | 1 | Hs.200412 |
| NM_001046 | SLC12A2 | −1 | Hs.162585 | NM_00145 | FOXC1 | −1 | Hs.348883 | NM_003289 | TPM2 | −1 | Hs.300772 |
| NM_014031 | SLC27A6 | −1 | Hs.49765 | NM_00145 | FOXF2 | −1 | Hs.484423 | AF109294 | MTAP | −1 | Hs.193268 |
| NM_016459 | PACAP | 1 | Hs.409563 | NM_01225 | HEY2 | −1 | Hs.144287 | AU145658 | MGC24103 | −1 | |
| AF151024 | PACAP | 1 | Hs.409563 | AI129628 | SAMD3 | 1 | Hs.558660 | NM_003558 | PIP5K1B | 1 | Hs.534371 |
| NM_033136 | FGF1 | −1 | Hs.483635 | AI633559 | MAP3K4 | −1 | Hs.390428 | AI949136 | COL27A1 | −1 | Hs.494892 |
| AB046841 | PCDHB16 | −1 | Hs.147674 | NM_00079 | DDC | −1 | Hs.359698 | AK021957 | COL27A1 | −1 | Hs.494892 |
| AL832028 | PCDHGA4 | −1 | | NM_01519 | COBL | −1 | Hs.99141 | BC005939 | PTGDS | 1 | Hs.558373 |
| AF231124 | SPOCK | −1 | Hs.124611 | BF855173 | SEP7 | 1 | Hs.191346 | NM_000954 | PTGDS | 1 | Hs.558373 |
| D13720 | ITK | 1 | Hs.558348 | M30894 | TRGC2 | 1 | | M61900 | PTGDS | 1 | Hs.558373 |
| N49841 | ZNF300 | −1 | Hs.134885 | M13231 | TRGC2 /// | 1 | Hs.534032 | NM_018159 | NUDT11 | −1 | Hs.200016 |
| NM_014211 | GABRP | −1 | Hs.26225 | M27331 | TRGC2 /// | 1 | Hs.534032 | AI308863 | CYBB | 1 | Hs.292356 |
| AL577024 | LOC22136 | 1 | Hs.7921 | M16768 | TRGC2 /// | 1 | Hs.534032 | AI436587 | P2RY8 | 1 | Hs.111377 |
| AB046819 | CPNE5 | 1 | Hs.372129 | BF792917 | HOXA10 | −1 | Hs.110637 | AI625739 | ARHGEF9 | −1 | Hs.54697 |
| NM_005084 | PLA2G7 | 1 | Hs.554780 | AI949827 | NFE2L3 | 1 | Hs.404741 | NM_014467 | SRPX2 | −1 | Hs.306339 |
| AI377755 | HLA-DQA1 | −1 | Hs.555872 | BE674103 | CROT | −1 | Hs.125039 | BF591534 | TCEAL7 | −1 | Hs.21861 |
| BE669692 | HLA-DRB1 | −1 | Hs.554754 | NM_01228 | KCND2 | −1 | Hs.21703 | NM_017938 | FAM70A | 1 | Hs.437563 |
| NM_002122 | HLA-DQA1 | 1 | Hs.387679 | NM_00240 | MEST | 1 | Hs.270978 | BE542323 | VGLL1 | −1 | Hs.496843 |
| K02403 | C4A /// C4 | 1 | Hs.546241 | NM_01325 | CLEC5A | −1 | Hs.446235 | NM_016267 | VGLL1 | −1 | Hs.496843 |
| AI435670 | SPDEF | 1 | Hs.485158 | AF495383 | ADAM9 | −1 | Hs.2442 | U10691 | MAGEA6 | 1 | Hs.441113 |
| AI583173 | HLA-DQB1 | 1 | Hs.409934 | NM_00622 | PNOC | 1 | Hs.88218 | BC000340 | MAGEA3 | 1 | Hs.417816 |
| BG397856 | HLA-DQA1 | 1 | Hs.387679 | NM_01447 | ADAMDE [symbol] | 1 | Hs.521459 | NM_005491 | CXorf6 | −1 | Hs.20136 |
| AL581873 | HLA-DOA | 1 | Hs.351874 | AF117949 | LOXL2 | −1 | Hs.116479 | | | | |

[symbol]

indicates data missing or illegible when filed

The IAP analysis, which included 141 genes, revealed that cell-to-cell signaling and interaction, cellular movement, antigen presentation, cell death, as well as cellular growth and proliferation were the most significant functional classes.

Both signatures were positively correlated with the ER and immune response modules and negatively correlated with the tumor invasion and angiogenesis modules, although the level of the correlation differs according to the signature (see Table 7).

TABLE 7
Spearman rho correlations between the de novo
signatures and the gene expression modules
Correlations Spearman's rho

| | | ER−/HER2+ | ER−/HER2− |
|---|---|---|---|
| | | signature | signature |
| ER−/HER2+ | Correlation Coefficient | 1 | 0.592219678 |
| signature | Sig. (2-tailed) | — | 0.000001 |
| | N | 118 | 118 |
| ER−/HER2− | Correlation Coefficient | 0.592219678 | 1 |
| signature | Sig. (2-tailed) | 0.000001 | — |
| | N | 118 | 118 |
| ER module | Correlation Coefficient | 0.491642289 | 0.367889007 |
| | Sig. (2-tailed) | 0.000001 | 4,15939E−05 |
| | N | 118 | 118 |
| HER2 module | Correlation Coefficient | 0.17831122 | 0.02552416 |
| | Sig. (2-tailed) | 0.053378276 | 0.783810651 |
| | N | 118 | 118 |
| Proliferation | Correlation Coefficient | −0.138255563 | −0.074315515 |
| module | Sig. (2-tailed) | 0.135428721 | 0.423834615 |
| | N | 118 | 118 |
| Tumor invasion | Correlation Coefficient | −0.287295622 | −0.378991231 |
| module | Sig. (2-tailed) | 0.001608367 | 2,31558E−05 |
| | N | 118 | 118 |
| Angiogenesis | Correlation Coefficient | −0.483739258 | −0.457137744 |
| module | Sig. (2-tailed) | 0.000001 | 0.000001 |
| | N | 118 | 118 |
| Immune | Correlation Coefficient | 0.695601839 | 0.43849039 |
| response | Sig. (2-tailed) | 0.000001 | 0.000001 |
| module | N | 118 | 118 |
| Apoptosis | Correlation Coefficient | 0.165083504 | 0.178501127 |
| module | Sig. (2-tailed) | 0.074025712 | 0.053120616 |
| | N | 118 | 118 |

**Correlation is significant at the 0.01 level (2-tailed).
*Correlation is significant at the 0.05 level (2-tailed).

Interestingly, when applying these signatures to the validation cohort, the ER−/HER2+ signature was predictive of pCR in the HER2+ patients treated in the anthracyclines arm [C-index: 0.75 (95% CI: 0.56-0.94), p=0.005], but not in the taxane arm, similarly the ER−/HER2− signature was only predictive of pCR in the HER2− patients treated with A but not with T chemotherapy [C-index: 0.65 (95% CI: 0.50-0.81), p=0.03]. The detailed results are given in Table 8.

TABLE 8
Results of the predictive ability of the de-novo
gene signatures, presented as C-indices, in the validation
cohort, according to treatment arm and HER2 mRNA status

| | | ER−/HER2− | ER−/HER2+ |
|---|---|---|---|
| | | signature | signature |
| A-arm | All ER− | 0.61 (0.48-0.74), | 0.61 (0.48-0.75), |
| | patients | p = 0.05 | p = 0.05 |
| | ER−/HER2− | 0.65(0.50-0.81), | 0.58 (0.43-0.74), |
| | patients | p = 0.03 | p = 0.15 |
| | ER−/HER2+ | 0.44 (0.21-0.66), | 0.75 (0.56-0.94), |
| | patients | p = 0.29 | p = 0.005 |
| T-arm | All ER− | 0.54 (0.39-0.69), | 0.52 (0.36-0.68), |
| | patients | p = 0.29 | p = 0.41 |
| | ER−/HER2− | 0.56 (0.39-0.73); | 0.56 (0.39-0.73); |
| | patients | p = 0.25 | p = 0.25 |
| | ER−/HER2+ | 0.47 (0.17-0.76), | 0.45 (0.14-0.76), |
| | patients | p = 0.41 | p = 0.38 |

The inventors further investigated the prognostic value of these signatures.

TABLE 9
Prognostic analysis of the “de novo” gene signatures

| | | | | |
|---|---|---|---|---|
| HR | lower 95 | higher 95 | p-value | n |

ALL PATIENTS

| | | | | | |
|---|---|---|---|---|---|
| ER−/HER2+ signature | 0.796 | 0.635 | 0.999 | 0.05 | 975 |
| ER−/HER2− signature | 0.88 | 0.702 | 1.102 | 0.27 | 975 |

ER−/HER2−

| | | | | | |
|---|---|---|---|---|---|
| ER−/HER2+ signature | 0.59 | 0.36 | 0.95 | 0.03 | 169 |
| ER−/HER2− signature | 0.93 | 0.6 | 1.46 | 0.76 | 174 |

HER2+

| | | | | | |
|---|---|---|---|---|---|
| ER−/HER2+ signature | 0.51 | 0.32 | 0.81 | 0.005 | 136 |
| ER−/HER2− signature | 0.39 | 0.18 | 0.84 | 0.02 | 136 |

ER+/HER2−

| | | | | | |
|---|---|---|---|---|---|
| ER−/HER2+ signature | 0.95 | 0.69 | 1.32 | 0.77 | 652 |
| ER−/HER2− signature | 1.02 | 0.76 | 1.39 | 0.88 | 652 |

ER+/HER2− L

| | | | | | |
|---|---|---|---|---|---|
| ER−/HER2+ signature | 0.92 | 0.47 | 1.78 | 0.8 | 335 |
| ER−/HER2− signature | 0.83 | 0.47 | 1.47 | 0.53 | 335 |

ER+/HER2−H

| | | | | | |
|---|---|---|---|---|---|
| ER−/HER2+ signature | 0.84 | 0.59 | 1.2 | 0.34 | 314 |
| ER−/HER2− signature | 0.84 | 0.59 | 1.18 | 0.31 | 314 |

As illustrated in FIG. 2 and in Table 9, high values of the ER−/HER2+ signature were associated with better prognosis in the global population, in the ER+/HER2− and HER2+ patients, whereas high values of the ER−/HER2− signature were only associated with better prognosis in HER2+ patients.

The inventors then combined the present invention with the results they previously obtained (Desmedt et al., 2008).

The inventors coupled the gene set of the invention, with a gene set comprising several genes from Table 10 (immune module 1).

| gene. symbol | EntrezGene. ID |
|---|---|
| ALPI | 248 |
| ANPEP | 290 |
| ARHGDIB | 397 |
| BAG4 | 9530 |
| BAX | 581 |
| BBS9 | 27241 |
| BID | 637 |
| BIRC3 | 330 |
| BLVRA | 644 |
| C17orf46 | 124783 |
| CASP10 | 843 |
| CASP6 | 839 |
| CASP8 | 841 |
| CASP9 | 842 |
| CD28 | 940 |
| CD33 | 945 |
| CD4 | 920 |
| CD40 | 958 |
| CD44 | 960 |
| CD5 | 921 |
| CD7 | 924 |
| CD80 | 941 |
| CD86 | 942 |
| CFLAR | 8837 |
| CR2 | 1380 |
| CRADD | 8738 |
| CSNK1D | 1453 |
| CUTL1 | 1523 |
| CYCS | 54205 |
| DAXX | 1616 |
| EIF4A1 | 1973 |
| EIF4E | 1977 |
| ELK1 | 2002 |
| FAF1 | 11124 |
| FAS | 355 |
| FKBP1A | 2280 |
| GRB2 | 2885 |
| HLA-A | 3105 |
| HLA-DRB1 | 3123 |
| HLA-DRB5 | 3127 |
| ICAM1 | 3383 |
| ICOSLG | 23308 |
| IKBKB | 3551 |
| IL10RA | 3587 |
| IL12B | 3593 |
| IL12RB2 | 3595 |
| IL13 | 3596 |
| IL15 | 3600 |
| IL1A | 3552 |
| IL2RA | 3559 |
| IL3 | 3562 |
| IL4R | 3566 |
| IRAK2 | 3656 |
| ITGA4 | 3676 |
| ITGAM | 3684 |
| ITGAX | 3687 |
| ITK | 3702 |
| JAK1 | 3716 |
| JAK3 | 3718 |
| JUNB | 3726 |
| LMNA | 4000 |
| LMNB1 | 4001 |
| LTA | 4049 |
| MADD | 8567 |
| MAF | 4094 |
| MAP2K3 | 5606 |
| MAP3K14 | 9020 |
| MAP3K7IP1 | 10454 |
| MAP4K2 | 5871 |
| MAPK1 | 5594 |
| MAPK8 | 5599 |
| MYD88 | 4615 |
| NCF2 | 4688 |
| NFKB1 | 4790 |
| NR3C1 | 2908 |
| NSMAF | 8439 |
| PAK2 | 5062 |
| PDK2 | 5164 |
| PIK3C2G | 5288 |
| PLCB1 | 23236 |
| PPP1R13B | 23368 |
| PPP3CA | 5530 |
| PRF1 | 5551 |
| PRKAR1B | 5575 |
| PRKDC | 5591 |
| PTEN | 5728 |
| PTENP1 | 11191 |
| PTPRC | 5788 |
| PVRL1 | 5818 |
| RAF1 | 5894 |
| RELA | 5970 |
| RHEB | 6009 |
| RPS6KB1 | 6198 |
| SPTAN1 | 6709 |
| STAT3 | 6774 |
| STAT5A | 6776 |
| TANK | 10010 |
| TAP1 | 6890 |
| TAP2 | 6891 |
| TGFB1 | 7040 |
| TNF | 7124 |
| TNFRSF10A | 8797 |
| TNFRSF13B | 23495 |
| TNFRSF1B | 7133 |
| TNFRSF25 | 8718 |
| TNFSF13B | 10673 |
| TOLLIP | 54472 |
| TRA@ | 6955 |
| TRAF1 | 7185 |
| TRAF3 | 7187 |

The inventors measured that increased expression of TOP2A index coupled with increased expression of genes of Table 10 increased the prognosis for chemotherapy (anthracyclines) and/or positively correlate with the response to chemotherapy (anthracyclines).

The inventors coupled the gene set of the invention, with a gene set comprising several genes from Table 11 (immune module 2).

| gene. symbol | EntrezGene. ID |
|---|---|
| ACP5 | 54 |
| ADAMDEC1 | 27299 |
| APOC1 | 341 |
| ARHGAP15 | 55843 |
| BIRC3 | 330 |
| BST2 | 684 |
| BTN3A2 | 11118 |
| CCL5 | 6352 |
| CCL8 | 6355 |
| CCRL2 | 9034 |
| CD2 | 914 |
| CD3G | 917 |
| CD40LG | 959 |
| CD48 | 962 |
| CD69 | 969 |
| CECR1 | 51816 |
| CLEC4A | 50856 |
| CTSC | 1075 |
| CXCL10 | 3627 |
| CXCL11 | 6373 |
| CXCL9 | 4283 |
| DDAH2 | 23564 |
| DDX58 | 23586 |
| DNAL4 | 10126 |
| EBI2 | 1880 |
| EBI3 | 10148 |
| ECGF1 | 1890 |
| EFNA1 | 1942 |
| ETV7 | 51513 |
| FASLG | 356 |
| FGL2 | 10875 |
| FLJ11286 | 55337 |
| FLJ20035 | 55601 |
| GLRX | 2745 |
| GPR171 | 29909 |
| GPR18 | 2841 |
| GZMK | 3003 |
| HCLS1 | 3059 |
| HCP5 | 10866 |
| HERC5 | 51191 |
| HERC6 | 55008 |
| IFI30 | 10437 |
| IFI44L | 10964 |
| IFI6 | 2537 |
| IFIT3 | 3437 |
| IFIT5 | 24138 |
| IFITM1 | 8519 |
| IGSF6 | 10261 |
| IL18 | 3606 |
| INDO | 3620 |
| IRF1 | 3659 |
| IRF8 | 3394 |
| ISG15 | 9636 |
| ITGB2 | 3689 |
| KLRC3 | 3823 |
| KLRK1 | 22914 |
| LAG3 | 3902 |
| LAMP3 | 27074 |
| LAPTM5 | 7805 |
| LGP2 | 79132 |
| LILRA4 | 23547 |
| LILRB1 | 10859 |
| MGC29506 | 51237 |
| MX1 | 4599 |
| MX2 | 4600 |
| NMI | 9111 |
| P2RX5 | 5026 |
| PIM2 | 11040 |
| PIP3-E | 26034 |
| PLA2G7 | 7941 |
| PLAC8 | 51316 |
| PSCDBP | 9595 |
| PSME1 | 5720 |
| PTPN7 | 5778 |
| RAB8A | 4218 |
| RASGRP1 | 10125 |
| REC8L1 | 9985 |
| RFX5 | 5993 |
| RSAD2 | 91543 |
| RTP4 | 64108 |
| SECTM1 | 6398 |
| SH2D1A | 4068 |
| SNX10 | 29887 |
| SP140 | 11262 |
| SPOCK2 | 9806 |
| STAT1 | 6772 |
| STAT4 | 6775 |
| TAP1 | 6890 |
| TFEC | 22797 |
| TRAF3 | 7187 |
| TRGV9 | 6983 |
| TRIM22 | 10346 |
| UBD | 10537 |
| VAV1 | 7409 |
| ZC3HAV1 | 56829 |

The inventors measured that increased expression of TOP2A index coupled with increased expression of genes of Table 11 increased the prognosis for chemotherapy (anthracyclines) and/or positively correlate with the response to chemotherapy (anthracyclines).

The inventors coupled the gene set of the invention, with a gene set comprising several genes from Table 12 (stroma module 1).

| gene. symbol | EntrezGene. ID | gene. symbol | EntrezGene. ID | gene. symbol | EntrezGene. ID |
|---|---|---|---|---|---|
| FGD6 | 55785 | LRP1B | 53353 | VIT | 5212 |
| PLAC9 | 219348 | TIMP4 | 7079 | HOP | 84525 |
| CAB39L | 81617 | STXBP6 | 29091 | GPX3 | 2878 |
| FGD6 | 55785 | WNT11 | 7481 | RRM2 | 6241 |
| LONRF3 | 79836 | PLAC9 | 219348 | GPX3 | 2878 |
| CGI-38 | 51673 | MICAL2 | 9645 | MYOC | 4653 |
| STXBP6 | 29091 | PKD1L2 | 114780 | CLEC3B | 7123 |
| FHL1 | 2273 | SDC1 | 6382 | GRP | 2922 |
| STXBP6 | 29091 | FHL1 | 2273 | GJB2 | 2706 |
| LEPR | 3953 | FHL1 | 2273 | AADAC | 13 |
| CA4 | 762 | F2RL2 | 2151 | MATN3 | 4148 |
| TNMD | 64102 | AKR1C2 | 1646 | PPAPDC1A | 196051 |
| POSTN | 10631 | LEF1 | 51176 | LOC646324 | 646324 |
| LOC58489 | 58489 | ADAM12 | 8038 | COL10A1 | 1300 |
| LOC284825 | 284825 | ADH1C | 126 | COL10A1 | 1300 |

The inventors measured that increased expression of TOP2A index coupled with reduced expression of genes of Table 12 increased the prognosis for chemotherapy (anthracyclines) and/or negatively correlate with the response to chemotherapy (anthracyclines).

The inventors coupled the gene set of the invention, with a gene set comprising several genes from Table 13: stroma module 2

| gene. symbol | EntrezGene. ID | gene. symbol | EntrezGene. ID | gene. symbol | EntrezGene. ID |
|---|---|---|---|---|---|
| PLAU | 5328 | BICD2 | 23299 | EPYC | 1833 |
| BMP1 | 649 | TNFRSF12A | 51330 | ANKRD46 | 157567 |
| MMP14 | 4323 | VDR | 7421 | CPNE1 | 8904 |
| THY1 | 7070 | SNAI2 | 6591 | BCL3 | 602 |
| COL5A2 | 1290 | EPB41L2 | 2037 | GLB1 | 2720 |
| ADAM12 | 8038 | FKBP14 | 55033 | UBL5 | 59286 |
| ANGPTL2 | 23452 | NBL1 | 4681 | ULK1 | 8408 |
| MFAP2 | 4237 | CAP1 | 10487 | NOL8 | 55035 |
| SERPINH1 | 871 | ATP6V1B2 | 526 | TGFB2 | 7042 |
| COL6A1 | 1291 | EPHB4 | 2050 | PDGFB | 5155 |
| ISLR | 3671 | TRAM2 | 9697 | BASP1 | 10409 |
| PDLIM7 | 9260 | DDR2 | 4921 | SDS | 10993 |
| PARVA | 55742 | GFPT2 | 9945 | RPS27A | 6233 |
| OLFML2B | 25903 | NID1 | 4811 | ENC1 | 8507 |
| TAGLN | 6876 | OFD1 | 8481 | ACAN | 176 |
| CTSA | 5476 | CADM1 | 23705 | ZNF518 | 9849 |
| PDGFRB | 5159 | STAB1 | 23166 | GPR89A | 51463 |
| MXRA8 | 54587 | TPST2 | 8459 | RPL18 | 6141 |
| OSMR | 9180 | PPP1R15A | 23645 | MEF2A | 4205 |
| COL3A1 | 1281 | PDLIM3 | 27295 | DNASE1L1 | 1774 |
| GREM1 | 26585 | ATPIF1 | 93974 | MYO1B | 4430 |
| FAP | 2191 | TRIM33 | 51592 | JPH2 | 57158 |
| DBN1 | 1627 | MMP3 | 4314 | | |

TABLE 14
ESR− module

| Gene ID | Gene Symbol | Gene ID | Gene Symbol | Gene ID | Gene Symbol | Gene ID | Gene Symbol |
|---|---|---|---|---|---|---|---|
| 2099 | ESR1 | 3295 | HSD17B4 | 2800 | GOLGA1 | 51103 | NDUFAF1 |
| 23158 | TBC1D9 | 11013 | TMSL8 | 8326 | FZD9 | 11042 | NA |
| 2625 | GATA3 | 51604 | PIGT | 6376 | CX3CL1 | 10040 | TOM1L1 |
| 771 | CA12 | 6663 | SOX10 | 8399 | PLA2G10 | 1117 | CHI3L2 |
| 3169 | FOXA1 | 85377 | MICALL1 | 5327 | PLAT | 112398 | EGLN2 |
| 4602 | MYB | 58495 | OVOL2 | 22885 | ABLIM3 | 9258 | MFHAS1 |
| 7802 | DNALI1 | 1116 | CHI3L1 | 11094 | C9orf7 | 374 | AREG |
| 18 | ABAT | 11001 | SLC27A2 | 5321 | PLA2G4A | 2982 | GUCY1A3 |
| 7494 | XBP1 | 25841 | ABTB2 | 57348 | TTYH1 | 688 | KLF5 |
| 57758 | SCUBE2 | 64080 | RBKS | 6787 | NEK4 | 1960 | EGR3 |
| 2066 | ERBB4 | 375035 | SFT2D2 | 123872 | LRRC50 | 7993 | UBXD6 |
| 9 | NAT1 | 10479 | SLC9A6 | 10421 | CD2BP2 | 25823 | TPSG1 |
| 10551 | AGR2 | 5002 | SLC22A18 | 5971 | RELB | 4485 | MST1 |
| 987 | LRBA | 8645 | KCNK5 | 6833 | ABCC8 | 23528 | ZNF281 |
| 56521 | DNAJC12 | 79885 | HDAC11 | 11122 | PTPRT | 1672 | DEFB1 |
| 2203 | FBP1 | 11254 | SLC6A14 | 23650 | TRIM29 | 28960 | DCPS |
| 51466 | EVL | 122616 | C14orf79 | 79629 | OCEL1 | 5268 | SERPINB5 |
| 51442 | VGLL1 | 79650 | C16orf57 | 8722 | CTSF | 934 | CD24 |
| 57496 | MKL2 | 23321 | TRIM2 | 57110 | HRASLS | 55450 | CAMK2N1 |
| 7031 | TFF1 | 23327 | NEDD4L | 6697 | SPR | 6261 | RYR1 |
| 1153 | CIRBP | 22977 | AKR7A3 | 2919 | CXCL1 | 2627 | GATA6 |
| 26227 | PHGDH | 8581 | LY6D | 27250 | PDCD4 | 57180 | ACTR3B |
| 1555 | CYP2B6 | 8842 | PROM1 | 23245 | ASTN2 | 4036 | LRP2 |
| 6648 | SOD2 | 4953 | ODC1 | 10265 | IRX5 | 29116 | MYLIP |
| 55638 | NA | 55544 | RBM38 | 2824 | GPM6B | 57211 | GPR126 |
| 221061 | C10orf38 | 55663 | ZNF446 | 10644 | IGF2BP2 | 4435 | CITED1 |
| 7033 | TFF3 | 27124 | PIB5PA | 7436 | VLDLR | 54913 | RPP25 |
| 53335 | BCL11A | 6715 | SRD5A1 | 25825 | BACE2 | 9982 | FGFBP1 |
| 79818 | ZNF552 | 51809 | GALNT7 | 10827 | C5orf3 | 11170 | FAM107A |
| 57613 | KIAA1467 | 89927 | C16orf45 | 4828 | NMB | 3294 | HSD17B2 |
| 8416 | ANXA9 | 1827 | DSCR1 | 6720 | SREBF1 | 6583 | SLC22A4 |
| 582 | BBS1 | 51706 | CYB5R1 | 10477 | UBE2E3 | 79170 | ATAD4 |
| 54463 | NA | 3383 | ICAM1 | 3066 | HDAC2 | 79745 | CLIP4 |
| 55733 | HHAT | 5806 | PTX3 | 55224 | ETNK2 | 2813 | GP2 |
| 2674 | GFRA1 | 9501 | RPH3AL | 875 | CBS | 6723 | SRM |
| 4478 | MSN | 3613 | IMPA2 | 3872 | KRT17 | 1360 | CPB1 |
| 51097 | SCCPDH | 7568 | ZNF20 | 753 | C18orf1 | 5016 | OVGP1 |
| 54502 | NA | 6280 | S100A9 | 136 | ADORA2B | 5271 | SERPINB8 |
| 26018 | LRIG1 | 22929 | SEPHS1 | 2013 | EMP2 | 347902 | AMIGO2 |
| 55793 | FAM63A | 81563 | C1orf21 | 1917 | EEF1A2 | 79719 | NA |
| 3868 | KRT16 | 1389 | CREBL2 | 3576 | IL8 | 55258 | NA |
| 54961 | SSH3 | 1410 | CRYAB | 419 | ART3 | 8563 | THOC5 |
| 60481 | ELOVL5 | 10884 | MRPS30 | 55650 | PIGV | 83464 | APH1B |
| 3667 | IRS1 | 55614 | C20orf23 | 23107 | MRPS27 | 23532 | PRAME |
| 83439 | TCF7L1 | 1824 | DSC2 | 25818 | KLK5 | 6834 | SURF1 |
| 10950 | BTG3 | 7851 | MALL | 8309 | ACOX2 | 6019 | RLN2 |
| 3572 | IL6ST | 2743 | GLRB | 1047 | CLGN | 214 | ALCAM |
| 4783 | NFIL3 | 427 | ASAH1 | 10002 | NR2E3 | 55333 | SYNJ2BP |
| 51161 | C3orf18 | 5241 | PGR | 60487 | TRMT11 | 10525 | HYOU1 |
| 2296 | FOXC1 | 51364 | ZMYND10 | 10656 | KHDRBS3 | 2232 | FDXR |
| 6664 | SOX11 | 6926 | TBX3 | 55240 | STEAP3 | 274 | BIN1 |
| 5613 | PRKX | 5193 | PEX12 | 3315 | HSPB1 | 10307 | APBB3 |
| 8543 | LMO4 | 8531 | CSDA | 10273 | STUB1 | 8986 | RPS6KA4 |
| 55686 | MREG | 23 | ABCF1 | 2171 | FABP5 | 56938 | ARNTL2 |
| 8100 | IFT88 | 7545 | ZIC1 | 55184 | C20orf12 | 9510 | ADAMTS1 |
| 2617 | GARS | 819 | CAMLG | 5783 | PTPN13 | 2770 | GNAI1 |
| 3945 | LDHB | 2947 | GSTM3 | 1877 | E4F1 | 4350 | MPG |
| 8382 | NME5 | 5825 | ABCD3 | 11098 | PRSS23 | 863 | CBFA2T3 |
| 10614 | HEXIM1 | 5860 | QDPR | 10202 | DHRS2 | 2891 | GRIA2 |
| 9633 | MTL5 | 59342 | SCPEP1 | 80223 | RAB11FIP1 | 10309 | UNG2 |
| 2568 | GABRP | 51806 | CALML5 | 79627 | OGFRL1 | 7037 | TFRC |
| 23324 | MAN2B2 | 79603 | LASS4 | 6948 | TCN2 | 3574 | IL7 |
| 55765 | C1orf106 | 21 | ABCA3 | 3097 | HIVEP2 | 55293 | UEVLD |
| 5104 | SERPINA5 | 54847 | SIDT1 | 8985 | PLOD3 | 27165 | GLS2 |
| 5174 | PDZK1 | 8537 | BCAS1 | 3892 | KRT86 | 55188 | RIC8B |
| 56674 | TMEM9B | 10874 | NMU | 10575 | CCT4 | 11202 | KLK8 |
| 1054 | CEBPG | 54149 | C21orf91 | 51004 | COQ6 | 51181 | DCXR |
| 9120 | SLC16A6 | 9929 | JOSD1 | 4071 | TM4SF1 | 827 | CAPN6 |
| 79641 | ROGDI | 5317 | PKP1 | 1718 | DHCR24 | 390 | RND3 |
| 23303 | KIF13B | 7388 | UQCRH | 1381 | CRABP1 | 54438 | GFOD1 |
| 2173 | FABP7 | 64764 | CREB3L2 | 9368 | SLC9A3R1 | 10079 | ATP9A |
| 23171 | GPD1L | 10127 | ZNF263 | 92104 | TTC30A | 4285 | MIPEP |
| 9674 | KIAA0040 | 80347 | COASY | 9518 | GDF15 | 8324 | FZD7 |
| 27134 | TJP3 | 126353 | C19orf21 | 6364 | CCL20 | 9052 | GPRC5A |
| 79921 | TCEAL4 | 50865 | HEBP1 | 3306 | HSPA2 | 9508 | ADAMTS3 |
| 54898 | ELOVL2 | 54812 | AFTPH | 79605 | PGBD5 | 10519 | CIB1 |
| 1345 | COX6C | 64087 | MCCC2 | 23336 | DMN | 7138 | TNNT1 |
| 5937 | RBMS1 | 8884 | SLC5A6 | 1356 | CP | 51735 | RAPGEF6 |
| 400451 | NA | 5269 | SERPINB6 | 54619 | CCNJ | 54970 | TTC12 |
| 3898 | LAD1 | 4321 | MMP12 | 9200 | PTPLA | 2591 | GALNT3 |
| 2530 | FUT8 | 8190 | MIA | 51302 | CYP39A1 | 2348 | FOLR1 |
| 51306 | C5orf5 | 6769 | STAC | 5191 | PEX7 | 2954 | GSTZ1 |
| 25837 | RAB26 | 51368 | TEX264 | 706 | TSPO | 23318 | ZCCHC11 |
| 10982 | MAPRE2 | 23541 | SEC14L2 | 7159 | TP53BP2 | 10267 | RAMP1 |
| 1632 | DCI | 9185 | REPS2 | 55218 | EXDL2 | 25984 | KRT23 |
| 7905 | REEP5 | 185 | AGTR1 | 79669 | C3orf52 | 6496 | SIX3 |
| 1101 | CHAD | 7368 | UGT8 | 10140 | TOB1 | 786 | CACNG1 |
| 323 | APBB2 | 399665 | FAM102A | 11226 | GALNT6 | 22976 | PAXIP1 |
| 28958 | CCDC56 | 12 | SERPINA3 | 6652 | SORD | 283232 | TMEM80 |
| 1476 | CSTB | 55975 | KLHL7 | 3418 | IDH2 | 629 | CFB |
| 9435 | CHST2 | 25864 | ABHD14A | 10200 | MPHOSPH6 | 7286 | TUFT1 |
| 7371 | UCK2 | 4851 | NOTCH1 | 7345 | UCHL1 | 5562 | PRKAA1 |
| 2737 | GLI3 | 9091 | PIGQ | 6564 | SLC15A1 | 9851 | KIAA0753 |
| 8685 | MARCO | 1299 | COL9A3 | 54458 | PRR13 | 79622 | C16orf33 |
| 55316 | RSAD1 | 83988 | NCALD | 60686 | C14orf93 | 23266 | LPHN2 |
| 6271 | S100A1 | 2920 | CXCL2 | 8792 | TNFRSF11A | 29104 | N6AMT1 |
| 55859 | BEX1 | 8870 | IER3 | 54894 | RNF43 | 1783 | DYNC1LI2 |
| 3595 | IL12RB2 | 55245 | C20orf44 | 5737 | PTGFR | 8987 | NA |
| 5100 | PCDH8 | 6666 | SOX12 | 1501 | CTNND2 | 79852 | ABHD9 |
| 2861 | GPR37 | 80279 | CDK5RAP3 | 7764 | ZNF217 | 57586 | SYT13 |
| 26278 | SACS | 1644 | DDC | 8405 | SPOP | 8785 | MATN4 |
| 55506 | H2AFY2 | 5441 | POLR2L | 1847 | DUSP5 | 10331 | B3GNT3 |
| 64215 | DNAJC1 | 9022 | CLIC3 | 4488 | MSX2 | 5357 | PLS1 |
| 3096 | HIVEP1 | 7769 | ZNF226 | 7163 | TPD52 | 54880 | BCOR |
| 23059 | CLUAP1 | 27239 | GPR162 | 25790 | CCDC19 | 55790 | NA |
| 79602 | ADIPOR2 | 26504 | CNNM4 | 5803 | PTPRZ1 | 4139 | MARK1 |
| 56683 | C21orf59 | 3400 | ID4 | 23635 | SSBP2 | 81539 | SLC38A1 |
| 22943 | DKK1 | 1733 | DIO1 | 6548 | SLC9A1 | 10810 | WASF3 |
| 6277 | S100A6 | 25915 | C3orf60 | 8187 | ZNF239 | 926 | CD8B |
| 65983 | GRAMD3 | 1525 | CXADR | 2588 | GALNS | 50805 | IRX4 |
| 4255 | MGMT | 1475 | CSTA | 54903 | MKS1 | 58513 | EPS15L1 |
| 10406 | WFDC2 | 2155 | F7 | 55163 | PNPO | 6304 | SATB1 |
| 3760 | KCNJ3 | 4188 | MDFI | 55101 | NA | 79446 | WDR25 |
| 23552 | CCRK | 3622 | ING2 | 4682 | NUBP1 | 23366 | NA |
| 9722 | NOS1AP | 25980 | C20orf4 | 3779 | KCNMB1 | 55699 | IARS2 |
| 23613 | PRKCBP1 | 8310 | ACOX3 | 64849 | SLC13A3 | | |
| 202 | AIM1 | 54820 | NDE1 | 4691 | NCL | | |
| 51207 | DUSP13 | 5816 | PVALB | 64428 | NARFL | | |

The inventors measured that increased expression of TOP2A index coupled with reduced expression of genes of Table 13 increased the prognosis for chemotherapy (anthracyclines) and/or negatively correlate with the response to chemotherapy (anthracyclines).

The inventors coupled the gene set of the invention, with a gene set comprising several genes from Table 14 (ESR−).

The inventors measured that increased expression of TOP2A index coupled with increased expression of genes of Table 14 increased the prognosis for chemotherapy (anthracyclines) and/or positively correlate with the response to chemotherapy (anthracyclines).

The inventors coupled the gene set of the invention, with a gene set comprising several genes from Table 15 and measured that increased expression of TOP2A index coupled with increased expression of genes of Table increased the prognosis for chemotherapy (anthracyclines) and/or positively correlate with the response to chemotherapy (anthracyclines).

TABLE 15
Erbb2 module

| Gene ID | Gene Symbol | Gene ID | Gene Symbol | Gene ID | Gene Symbol | Gene ID | Gene Symbol |
|---|---|---|---|---|---|---|---|
| 2064 | ERBB2 | 147179 | WIPF2 | 1573 | CYP2J2 | 404093 | CUEDC1 |
| 93210 | PERLD1 | 55040 | EPN3 | 26154 | ABCA12 | 3675 | ITGA3 |
| 5709 | PSMD3 | 5245 | PHB | 3081 | HGD | 55129 | TMEM16K |
| 5409 | PNMT | 9635 | CLCA2 | 8804 | CREG1 | 24147 | FJX1 |
| 55876 | GSDML | 3227 | HOXC11 | 9914 | ATP2C2 | 1048 | CEACAM5 |
| 22794 | CASC3 | 29095 | ORMDL2 | 5129 | PCTK3 | 9572 | NR1D1 |
| 3927 | LASP1 | 5909 | RAP1GAP | 54793 | KCTD9 | 51375 | SNX7 |

The inventors coupled the gene set of the invention, with a gene set comprising several genes from Table 16.

TABLE 16
AURKA proliferation module

| Gene ID | Gene Symbol | Gene ID | Gene Symbol | Gene ID | Gene Symbol | Gene ID | Gene Symbol |
|---|---|---|---|---|---|---|---|
| 6790 | AURKA | 2331 | FMOD | 51204 | CCDC44 | 3028 | HSD17B10 |
| 11065 | UBE2C | 51512 | GTSE1 | 54845 | RBM35A | 26519 | TIMM10 |
| 9133 | CCNB2 | 6424 | SFRP4 | 283 | ANG | 25960 | GPR124 |
| 1058 | CENPA | 55353 | LAPTM4B | 79652 | C16orf30 | 10252 | SPRY1 |
| 332 | BIRC5 | 8404 | SPARCL1 | 56944 | OLFML3 | 6199 | RPS6KB2 |
| 11004 | KIF2C | 990 | CDC6 | 3297 | HSF1 | 9824 | ARHGAP11A |
| 10112 | KIF20A | 7043 | TGFB3 | 27235 | COQ2 | 55630 | SLC39A4 |
| 991 | CDC20 | 11047 | ADRM1 | 2487 | FRZB | 7049 | TGFBR3 |
| 2305 | FOXM1 | 58190 | CTDSP1 | 3251 | HPRT1 | 8607 | RUVBL1 |
| 891 | CCNB1 | 79838 | TMC5 | 5119 | PCOLN3 | 2581 | GALC |
| 22974 | TPX2 | 84823 | LMNB2 | 6839 | SUV39H1 | 862 | RUNX1T1 |
| 9088 | PKMYT1 | 83989 | C5orf21 | 27303 | RBMS3 | 8458 | TTF2 |
| 54478 | FAM64A | 1793 | DOCK1 | 10468 | FST | 9775 | EIF4A3 |
| 4751 | NEK2 | 9358 | ITGBL1 | 26289 | AK5 | 3181 | HNRPA2B1 |
| 24137 | KIF4A | 8836 | GGH | 55038 | CDCA4 | 26039 | SS18L1 |
| 23397 | NCAPH | 57088 | PLSCR4 | 7283 | TUBG1 | 10580 | SORBS1 |
| 9319 | TRIP13 | 6642 | SNX1 | 23212 | RRS1 | 7056 | THBD |
| 4085 | MAD2L1 | 4969 | OGN | 65094 | JMJD4 | 8322 | FZD4 |
| 9156 | EXO1 | 90627 | STARD13 | 55379 | LRRC59 | 1003 | CDH5 |
| 10615 | SPAG5 | 11260 | XPOT | 10956 | NA | 2152 | F3 |
| 7083 | TK1 | 22827 | NA | 51022 | GLRX2 | 55068 | NA |
| 6491 | STIL | 9793 | CKAP5 | 54915 | YTHDF1 | 64785 | GINS3 |
| 6241 | RRM2 | 2791 | GNG11 | 54861 | SNRK | 79042 | TSEN34 |
| 55839 | CENPN | 55247 | NEIL3 | 79000 | C1orf135 | 8805 | TRIM24 |
| 7298 | TYMS | 10234 | LRRC17 | 79776 | ZFHX4 | 1478 | CSTF2 |
| 641 | BLM | 9353 | SLIT2 | 79971 | GPR177 | 1746 | DLX2 |
| 4171 | MCM2 | 1841 | DTYMK | 7718 | ZNF165 | 57125 | PLXDC1 |
| 1164 | CKS2 | 9631 | NUP155 | 201254 | STRA13 | 22998 | NA |
| 79682 | MLF1IP | 5424 | POLD1 | 1848 | DUSP6 | 79915 | C17orf41 |
| 10129 | FRY | 6631 | SNRPC | 9037 | SEMA5A | 7026 | NR2F2 |
| 51659 | GINS2 | 10186 | LHFP | 5433 | POLR2D | 7474 | WNT5A |
| 10212 | DDX39 | 4521 | NUDT1 | 29087 | THYN1 | 55857 | C20orf19 |
| 3925 | STMN1 | 3479 | IGF1 | 79864 | C11orf63 | 114625 | ERMAP |
| 79801 | SHCBP1 | 4172 | MCM3 | 358 | AQP1 | 8857 | FCGBP |
| 3014 | H2AFX | 2205 | FCER1A | 6634 | SNRPD3 | 26872 | STEAP1 |
| 10535 | RNASEH2A | 55732 | C1orf112 | 2621 | GAS6 | 7226 | TRPM2 |
| 5984 | RFC4 | 9077 | DIRAS3 | 56270 | WDR45L | 29844 | TFPT |
| 55970 | GNG12 | 5557 | PRIM1 | 5187 | PER1 | 4719 | NDUFS1 |
| 1033 | CDKN3 | 54963 | UCKL1 | 2098 | ESD | 4013 | LOH11CR2A |
| 55388 | MCM10 | 54512 | EXOSC4 | 81887 | LAS1L | 3396 | ICT1 |
| 55257 | C20orf20 | 79901 | CYBRD1 | 1811 | SLC26A3 | 397 | ARHGDIB |
| 1163 | CKS1B | 10161 | P2RY5 | 54535 | CCHCR1 | 10436 | EMG1 |
| 8914 | TIMELESS | 29097 | CNIH4 | 55526 | DHTKD1 | 51582 | AZIN1 |
| 54821 | NA | 6513 | SLC2A1 | 57161 | PELI2 | 10598 | AHSA1 |
| 23371 | TENC1 | 51123 | ZNF706 | 2353 | FOS | 333 | APLP1 |
| 8544 | PIR | 857 | CAV1 | 51279 | C1RL | 51142 | CHCHD2 |
| 8317 | CDC7 | 51110 | LACTB2 | 60436 | TGIF2 | 27123 | DKK2 |
| 55020 | NA | 58500 | ZNF250 | 4649 | MYO9A | 594 | BCKDHB |
| 23460 | ABCA6 | 11081 | KERA | 53820 | DSCR6 | 6286 | S100P |
| 64321 | SOX17 | 7064 | THOP1 | 3638 | INSIG1 | 3954 | LETM1 |
| 7098 | TLR3 | 55799 | CACNA2D3 | 11171 | STRAP | 51087 | YBX2 |
| 6338 | SCNN1B | 49855 | ZNF291 | 10992 | SF3B2 | 10953 | TOMM34 |
| 3692 | ITGB4BP | 54606 | DDX56 | 6832 | SUPV3L1 | 8317 | CDC7 |
| 10253 | SPRY2 | 7164 | TPD52L1 | 55922 | NKRF | 51110 | LACTB2 |
| 2669 | GEM | 80775 | TMEM177 | 10557 | RPP38 | 60436 | TGIF2 |
| 79679 | VTCN1 | 667 | DST | 3216 | HOXB6 | 27123 | DKK2 |
| 79618 | HMBOX1 | 2781 | GNAZ | 54785 | C17orf59 | | |
| 8772 | FADD | 23464 | GCAT | 1933 | EEF1B2 | | |
| 9986 | RCE1 | 79763 | ISOC2 | 8161 | COIL | | |

The inventors measured that increased expression of TOP2A index coupled with increased expression of genes of Table 16 increased the prognosis for chemotherapy (anthracyclines) and/or positively correlate with the response to chemotherapy (anthracyclines).

The inventors coupled the gene set of the invention, with a gene set comprising several genes from Table 17 (VEGF).

| Entrez | Gene | Entrez | Gene | Entrez | Gene |
|---|---|---|---|---|---|
| Gene ID | Symbol | Gene ID | Symbol | Gene ID | Symbol |
| 7422 | VEGFA | 6166 | RPL36AL | 22809 | ATF5 |
| 911 | CD1C | 9450 | LY86 | 23417 | MLYCD |
| 4005 | LMO2 | 22900 | CARD8 | 23592 | LEMD3 |
| 4222 | MEOX1 | 1776 | DNASE1L3 | 51621 | KLF13 |
| 29927 | SEC61A1 | 1119 | CHKA | | |

The inventors coupled the gene set of the invention, with a gene set comprising several genes from Table 18 (CASP3).

| Gene ID | Gene Symbol | Gene ID | Gene Symbol | Gene ID | Gene Symbol | Gene ID | Gene Symbol |
|---|---|---|---|---|---|---|---|
| 836 | CASP3 | 7738 | ZNF184 | 8237 | USP11 | 25978 | CHMP2B |
| 10393 | ANAPC10 | 3728 | JUP | 402 | ARL2 | 6301 | SARS |
| 55361 | NA | 5977 | DPF2 | | | | |

### Methods

### Patients

### Study Population

The neoadjuvant prospective “TOP” (Trial of Principle) trial was conducted at different European hospitals and coordinated by the Institut Jules Bordet. This study is registered on the clinical trials site of the US National Cancer Institute website http://clinicaltrials.gov/ct2/show/NCT00162812?term=NCT00162812&rank=1. One hundred and forty-nine patients have been included in this trial. Anthracyclines (Epirubicin) monotherapy (100 mg/m2) was given as neo-adjuvant chemotherapy: every 3 weeks×4 cycles for early BC or every 2 weeks×6 cycles with Neulasta® 6 mg on day 2 for patients with inflammatory or locally advanced breast cancer. At completion of chemotherapy, every patient underwent surgery with axillary node sampling. After surgery, adjuvant docetaxel (100 mg/m2×4 cycles) and loco-regional irradiation were administered using standard criteria. This study was primarily designed for the identification of biomarkers of response to anthracyclines (epirubicin).

All patients underwent pretreatment core biopsies of the primary breast tumor before starting neo-adjuvant chemotherapy using a 14G needle. Two biopsies were embedded in OCT (Sakura), frozen in liquid nitrogen within 5 minutes and transferred to a −80° C. freezer. Two biopsies were fixed in formalin and embedded in paraffin. Both fixed and frozen samples were retrieved and stored at the Institut Jules Bordet in Brussels, where the TOP2A evaluations were carried out. Pathologic response was determined by microscopic examination of the excised tumor and nodes after completion of chemotherapy. Pathological complete response (pCR) was defined by the absence of residual invasive breast carcinoma (macro and microscopic) in the breast and in the axillary nodes. Persistence of in-situ carcinoma without invasive component was considered pCR.

This analysis was performed after the clinical data until surgery had become available for all the patients. The clinical data was collected, monitored and validated by the BrEAST data centre, Institut Jules Bordet. This study has been approved by the local ethics committees and all patients have given written informed consent prior to study entry.

### Validation Cohort

This validation cohort includes patients reported by Bonnefoi et al. These patients are a subgroup of the patients included in the prospective phase III intergroup trial of neoadjuvant chemotherapy (European Organisation for Research and Treatment of Cancer [EORTC] 10994/Breast International Group [BIG] 00-01, registered as NCT00017095).

Patients were randomly assigned to a non-taxane regimen of six cycles of 500 mg/m² fluorouracil, 100 mq/m² anthracyclines (epirubicin), and 500 mg/m² cyclophosphamide (FEC) treatment, or to three cycles of 100 mg/m² docetaxel followed by three cycles of 90 mg/m² anthracyclines (epirubicin) plus 70 mg/m² docetaxel (TET). This substudy was restricted to ER-negative tumours. The definition used this study for pCR was slightly different than the one used in our study. It was defined as disappearance at the invasive component of the primary tumour after treatment, with at most a few scattered tumour cells detected by the pathologist in the resection specimen, but did not consider the disappearance of the invasive component in the axillary lymph nodes. Microarray data were deposited in the Gene Expression Omnibus database under accession number GSE6861.

### Fluorescent Hybridization In Situ (FISH)

FISH assays were done with the Abbott Multi-color TOP2A Spectrum orange, HER2 Spectrum green and CEP17 Spectrum aqua probe. Briefly, the sections were deparaffined and incubated in pre-treatment buffer at 80° C. for 30 min. Enzymatic digestion was carried out with pepsin (10-20 min at 37° C.) and the slides were dehydrated in graded dilutions of ethanol. The probe (10 μl) was applied to the slides under coverslips. The slides were co-denatured on a hot plate (73° C. for 5 min), followed by overnight hybridization at 37° C. After stringency washing (2×SSC/0.3% Nonidet P-40 at 73° C. for 2 min), the slides were counterstained with 10 μl of 0.2 μM 4,6-diamino-2-phenylindole (DAPI) in antifade solution (Vectashield, Vector Laboratories, Inc., Burlingame, Calif.). FISH was evaluated using an Olympus BX51 epifluorescence microscope. The invasive part of the tumor was circled on the slides with a diamond by superposition with haematoxylin-stained sections previously analyzed by the pathologist (DL). Signals from at least 60 non-overlapping nuclei with intact morphology were evaluated to determine the mean number of signals/cell (ratio between mean number of TOP2A or HER2 signals and the mean

### Immunohistochemistry (IHC)

TOP2A protein expression was evaluated by IHC. Briefly, the sections were dewaxed, rehydrated and incubated for 30 min in 0.5% hydrogen peroxide (H₂O₂) in methanol. After pretreatment (0.1% trypsine in 0.1% CaCl2 pH 7.8 for 10 min at 37° C.), non-specific staining was blocked by incubating in 10% normal serum for 1 h at 4° C. and performing all steps in buffer PBS/0.1% BSA/1% Tween-20. After incubation with the primary antibody (1 μg/ml overnight at 4° C., clone KiS1, Boehringer-Mannheim), sections were incubated at room temperature with a secondary biotinylated anti-mouse antibody for 30 min and Streptavidin-HRP (Zymed) (1/20) for 10 min. 3′3-diaminobenzidine was used as a chromogen. Sections were counterstained with Mayer's haematoxylin. Negative controls consisted in serial sections incubated with buffer alone instead of primary antibody. Tonsil samples were used as positive control.

Ki-67 evaluation was carried out routinely by IHC using the monoclonal mouse antibody MIB-1 (1/50, Dako, Carpinteria, Calif.). In brief, the sections were dewaxed and rehydrated. Then a microwave (two times 10 min at 650 W) antigen retrieval method in citrate buffer pH 6.0 was implemented before using the Ventana Nexes automated immunostainer with standard Nexes reagents (Ventana Medical Systems, Tucson, Ariz.). A cutoff of 25% of positively stained cells was used (Durbecq et al. 2004).

### Gene Expression Profiling

One 5-μm tissue section (usually after 10 30-μm sections) of each biopsy were hematoxylin and eosin stained to monitor the tumor cell percentage of the tissue. Only specimens with more than 30% of tumor cells were included in further analysis. Isolation of RNA was performed using the Trizol method (Invitrogen) according to the manufacturer's instructions and purified using RNeasy mini-columns (Qiagen, Valencia, Calif.). The quality of the RNA obtained from each tumor sample was assessed based on the RNA profile generated by the bioanalyzer (Agilent Inc). RNA amplification, hybridization and image scanning were done according to standard Affymetrix protocols. The inventors have used the Affymetrix Human Genome U133-2.0 plus GeneChip.

### Statistical Analysis

Correlations between continuous variables were assessed using the non-parametric Spearman coefficients. Correlations between binary variables were reported using the kappa statistics. Other correlations between categorical variables were performed using the chi-square test. Correlations between continuous and categorical variables were performed using the Mann-Withney U (for binary variables) or Kruskal-Wallis test.

Both in the study population and validation cohort, HER2+ and HER2− patients were identified using the bimodality of the HER2 mRNA expression as previously described.

The TOP2A index and other prognostic indexes were computed for each sample as

$$module score = {\sum }_{i} {w}_{i} {x}_{i} / {\sum }_{i} \vert {w}_{i} \vert$$

where xᵢ is the expression of a gene included in the index and wᵢ is its coefficient. This index is a combination of table 1 genes.

More precisely, the inventors used an index made of the preferred genes: TOP2A, THRA, CDC6 and RARA. Alternatively, the inventors tested CDC6, RARA and WIPF2 with even an improved diagnostic.

When different probe-sets were available for one gene, the inventors considered the one with the greatest variance (THRA: 31637_s_at, CDC6: 203 967_at, RARA: 203 749_s_at and TOP2A: 201 291_s_at).

The signatures predictive of pathological response were identified through stability-based feature ranking as scoring function.

Once the signature was identified, a signature score was computed for each patient using the following formula:

$$s = {\sum }_{i} {w}_{i} {x}_{1} / {\sum }_{i} \vert {w}_{i} \vert$$

where s is the signature score, xᵢ is the expression of a gene in the signature, wᵢ is either +1 or −1 depending on the sign of the association with pathological response. The signature scores were scaled such that quantiles 2.5% and 97.5% are equaled to −1 and +1 respectively. This scaling is robust to outliers and ensured that the scores lay approximately in [−1,+1].

The area under the curve (AUC) was used to assess the prediction performance of a signature score. AUC was estimated through the concordance index, its confidence interval and significance being estimated assuming asymptotic normality. The corresponding p-values were one-sided.

The inventors computed the prognostic value of a signature score through a meta-analytical framework.

The corresponding gene module scores were computed in order to identify the breast cancer molecular subtype in this two dimensional space using a mixture of three Gaussians. The three subtypes are denoted by ER−/HER− (basal), HER2+ (ERBB2-enriched) and ER+/HER2− (luminal).

### Functional Analysis

Functional analysis of the gene signatures was performed using Ingenuity Pathways Analysis (IPA) tools version 3.0. Affymetrix probe sets of each cluster were used as input and IPA then calculated a significance value for enrichment of the functional classes. Only significant functions are shown.

1. A gene set comprising 2, 3, 4, 5, 6, 7, 8, 9, 10, 15, 16, 17, 18, 19, 20, 21, 22 or all the genes of Table 1.

2. The gene set of claim 1 comprising CDC6 (Gene ID: 990), RARA (Gene ID: 5914) and WIPF2 (Gene ID: 147179).

3. The gene set of claim 1 consisting of CDC6 (Gene ID: 990), RARA (Gene ID: 5914) and WIPF2 (Gene ID: 147179).

4. The gene set of claim 1 comprising THRA (Gene ID: 9572), CDC6 (Gene ID: 990), RARA (Gene ID: 5914) and TOP2A (Gene ID: 7153).

5. The gene set of claim 1 consisting of THRA (Gene ID: 9572), CDC6 (Gene ID: 990), RARA (Gene ID: 5914) and TOP2A (Gene ID: 7153).

6. The gene set according to claim 2 further comprising 1, 2, 3, 4, 5, 6, 7, 8, 9, 10, 15, 16, 17, 18 or all the (other) genes of Table 1.

7. The gene set according to claim 1 further comprising FAM64A, KIF4A, NCAPH and STIL.

8. The gene set according to claim 1 consisting of CDC6 (Gene ID: 990), RARA (Gene ID: 5914), WIPF2 (Gene ID: 147179), FAM64A, KIF4A, NCAPH and STIL.

9. The gene set according to claim 1, further comprising 1, 2, 3, 4, 5, 6, 7, 8, 9, 10, 15, 20, 25, 30, 35, 40, 45, 50, 55, 60, 65, 70, 75 or all the genes of Table 5 and/or Table 6.

10. The gene set according to claim 1, further comprising 1, 2, 3, 4, 5, 6, 7, 8, 9, 10 or all the genes of Table 10 and/or of Table 11.

11. The gene set according to claim 1 further comprising 1, 2, 3, 4, 5, 6, 7, 8, 9, 10 or all the genes of Table 12 and/or of Table 13.

12. The gene set according to claim 1 further comprising 1, 2, 3, 4, 5, 6, 7, 8, 9, 10 or all the genes of Table 11 and/or 1, 2, 3, 4, 5, 6, 7, 8, 9, 10 or all the genes of Table 13.

13. The gene set according to claim 1 further comprising 1, 2, 3, 4, 5, 6, 7, 8, 9, 10 or all the genes of Table 14.

14. The gene set according to claim 1 further comprising 1, 2, 3, 4, 5, 6, 7, 8, 9, 10 or all the genes of Table 15.

15. The gene set according to claim 1 further comprising 1, 2, 3, 4, 5, 6, 7, 8, 9, 10 or all the genes of Table 16.

16. A diagnostic kit or device comprising, fixed upon a solid support, capture probes detecting specifically the expression of target genes of the gene set according to claim 1 and possibly other means for real time PCR analysis.

17. A diagnostic kit or device comprising, fixed upon a solid support, capture probes detecting specifically the expression of target genes of a gene set comprising 2, 3, 4, 5, 6, 7, 8, 9, 10, 15, 20, 25, 30, 35, 40, 45; 50, 55, 60, 65, 70, 75 or all the genes of Table 5 and possibly other means for real time PCR analysis.

18. A diagnostic kit or device comprising, fixed upon a solid support, captures probes detecting specifically the expression of target genes of a gene set comprising 2, 3, 4, 5, 6, 7, 8, 9, 10, 15, 20, 25, 30, 35, 40, 45, 50, 55, 60, 65, 70, 75 or all the genes of Table 6 and possibly other means for real time PCR analysis.

19. The kit or device according to claim 17 being a computerized system comprising
a bio-assay module configured for detecting a gene expression or protein synthesis from a tumor sample (a breast tumor sample) based upon the gene set, kit or device having a gene set comprising 2, 3, 4, 5, 6, 7, 8, 9, 10, 15, 16, 17, 18, 19, 20, 21, 22 or all the genes of Table 1,
a processor module configured to calculate expression of the said genes expression or the said protein synthesis and to generate a risk assessment for the tumor, preferably breast tumor sample.

20. A method for prediction of cancer in mammal subject, which comprises the step of measuring gene expression in a (breast) tumor sample obtained from the said mammal subject, by putting into contact nucleotide sequences obtained from this tumor sample with the gene set, the kit or the device according to any of the preceding claims and possibly generating a risk assessment for the said tumor sample by designating the outcome of a regimen comprising administration to the said mammal subject, of one or more Anthracycline compound(s).

21. The method of claim 20 wherein the patient is a human patient.

22. The method of claim 21, wherein the patient is suffering from a breast cancer.

23. The method of claim 22, wherein the patient is suffering from ER− breast cancer.

24. The method according to claim 20, wherein the patient is suffering from Her2+ breast cancer.

25. An Anthracycline compound for use in the treatment and/or the prevention of cancer having an increased risk assessment as measured by the method according to claim 20, and by the gene set, kit or device having a gene set comprising 2, 3, 4, 5, 6, 7, 8, 9, 10, 15, 16, 17, 18, 19, 20, 21, 22 or all the genes of Table 1.

26. An Anthracycline compound or a mixture of Anthracycline compounds for use in the treatment and/or the prevention of a (breast) cancer having an increased prognosis as measured by the method according to claim 20, and by the gene set, kit or device having a gene set comprising 2, 3, 4, 5, 6, 7, 8, 9, 10, 15, 16, 17, 18, 19, 20, 21, 22 or all the genes of Table 1.
